(19) United States
(12) Patent Application Publication
(10) Pub. No.: US 2025/0037800 A1
(43) Pub. Date: Jan. 30, 2025

(54) METHODS AND SYSTEMS FOR IDENTIFYING GENES ASSOCIATED WITH BIOSYNTHETIC GENE CLUSTERS

(71) Applicant: LifeMine Therapeutics, Inc., Cambridge, MA (US)

(72) Inventors: Michalis HADJITHOMAS, Cambridge, MA (US); Stephen Andrew WYKA, Cambridge, MA (US); Jinwoo KIM, Cambridge, MA (US); Yu-Cheng LIN, Cambridge, MA (US); Iain James MCFADYEN, Cambridge, MA (US); Gregory L. VERDINE, Cambridge, MA (US)

(21) Appl. No.: 18/654,823

(22) Filed: May 3, 2024

Related U.S. Application Data
(63) Continuation of application No. PCT/US2022/049016, filed on Nov. 4, 2022.
(60) Provisional application No. 63/278,065, filed on Nov. 10, 2021.
(60) Provisional application No. 63/263,638, filed on Nov. 5, 2021.

(51) Int. Cl. G16B 40/20

(52) U.S. Cl. CPC G16B 40/20

(57) ABSTRACT

The present disclosure relates to methods and systems for identifying genes associated with biosynthetic gene clusters (BGCs), including embedded target genes (ETaGs) that are homologs of potential therapeutic targets. The methods and systems described herein apply a comparative genomics and manual review or machine-learning model to analyze grid representations such as heat maps, which assess across a plurality of diverse genomes distribution of orthologs (e.g., bidirectional best hits) of a plurality of query genes that are co-localized with an anchor gene (e.g., a core synthase gene) of a BGC in a query genome.

### CROSS-REFERENCE TO RELATED APPLICATIONS

This application is a continuation of International Application No. PCT/US2022/049016, which designated the United States and was filed on Nov. 4, 2022, published in English, which claims the priority benefit of U.S. Provisional Patent Application Ser. No. 63/263,638, filed Nov. 5, 2021, and the priority benefit of U.S. Provisional Patent Application Ser. No. 63/278,065, filed Nov. 10, 2021, the contents of each of which are incorporated herein by reference in their entirety.

### FIELD

The present disclosure relates generally to methods and systems for identifying genes associated with gene clusters (e.g., biosynthetic gene clusters), and applications thereof including methods of determining the boundaries of gene clusters (e.g., the boundaries of biosynthetic gene clusters), methods for identifying therapeutic targets, and methods for drug discovery.

### BACKGROUND

Microbes produce a wide variety of small molecule compounds, known as secondary metabolites or natural products, which have diverse chemical structures and functions. Some secondary metabolites allow microbes to survive adverse environments, while others serve as weapons of inter- and intra-species competition. See, e.g., Piel, J. Nat. Prod. Rep., 26:338-362, 2009. Many human medicines (including, e.g., antibacterial agents, antitumor agents, and insecticides) have been derived from secondary metabolites. See, e.g., Newman D. J. and Cragg G. M. J. Nat. Prod., 79:629-661, 2016.

Microbes synthesize secondary metabolites using enzyme proteins encoded by clusters of co-located genes called biosynthetic gene clusters (BGCs). Evidence is emerging that some microbial biosynthetic gene clusters contain genes that appear not to be involved in synthesis of the relevant biosynthetic products produced by the enzymes encoded by the clusters. In some cases, such non-biosynthetic genes have been described as “self-protective” because they encode proteins that apparently can render the host organism resistant to the relevant biosynthetic product. For example, in some cases, non-biosynthetic genes encoding transporters of the biosynthetic products, detoxification enzymes that act on the biosynthetic products, or resistant variants of proteins whose activities are targeted by the biosynthetic products, have been reported. See, for example, Cimermancic, et al., Cell 158:412, 2014; Keller, Nat. Chem. Biol. 11:671, 2015. Researchers have proposed that identification of such genes, and determination of their functions, could be useful in determining the role of the biosynthetic products synthesized by the enzymes of the clusters. See, for example, Yeh, et al., ACS Chem. Biol. 11:2275, 2016; Tang, et al., ACS Chem. Biol. 10:2841, 2015; Regueira, et al., Appl, Environ. Microbiol. 77: 3035, 2011; Kennedy, et al., Science 284:1368, 1999; Lowther, et al., Proc. Natl. Acad. Sci. USA 95:12153, 1998; Abe, et al., Mol. Genet. Genomics 268:130, 2002. United States Patent Application Publication No. 2020/0211673 A1 provides insights that certain non-biosynthetic genes present in biosynthetic gene clusters, or present in close proximity to the biosynthetic genes of the clusters (particularly in eukaryotic, e.g., fungal, biosynthetic gene clusters as contrasted with bacterial biosynthetic gene clusters) may represent homologs of human genes that are targets of therapeutic interest. Such non-biosynthetic genes are referred to as “embedded target genes” or “ETaGs.”

Traditionally, secondary metabolites have been identified from microbial cultures and screened for therapeutic activities against human targets of interest. However, the vast majority of microbes are not culturable, and even BGCs in culturable microbes can remain transcriptionally silent under laboratory conditions. Recent developments in nucleic acid and protein sequencing technologies and bioinformatics pipelines have enabled rapid identification of a large number of BGCs from environmental microbes without having to culture the microbes and test the bioactivity of the BGCs. See, e.g., Palazzotto E. and Weber T., Curr. Opin. Microbiol., 45:109-116, 2018. However, it remains a challenge to precisely define the genomic boundaries of BGCs using purely computational methods. There are also no computational pipelines available to identify embedded genes in BGCs that confer self-protection against the secondary metabolites produced by the BGCs.

### SUMMARY

Disclosed herein are exemplary methods, systems, and non-transitory storage media for identifying genes associated with (or embedded within) a gene cluster (e.g., a biosynthetic gene cluster (BGC) involved in the synthesis of primary or secondary metabolites). The disclosed methods and systems may be used for determining the boundaries of a gene cluster (e.g., the boundaries of a BGC) in a genome, identifying an embedded gene of the cluster, identifying a resistance gene against a secondary metabolite produced by a BGC, or for assisting in the identification of a small molecule modulator of a target gene (i.e., a gene of interest) or a protein encoded by the target gene.

One aspect of the present application provides a computer-implemented method for determining a likelihood that a putative embedded gene is associated with a gene cluster (e.g., a BGC), wherein the putative embedded gene is co-localized with an anchor gene (e.g., a core synthase gene) known to associate with the gene cluster (e.g., the BGC) in a query genome, comprising: a) receiving a grid representation (e.g., a heat map) comprising a plurality of cells arranged according to a first axis and a second axis, wherein the first axis corresponds to a plurality of different genomes, wherein the second axis corresponds to a plurality of query genes that are co-localized with the anchor gene (e.g., core synthase gene) of the gene cluster (e.g., the BGC) in the query genome, wherein the putative embedded gene is one of the plurality of query gene orthologs, and wherein each cell is based on: (i) the presence or absence of an ortholog (e.g., a bidirectional best hit or “BBH”) of the respective query gene in the respective genome; (ii) sequence similarity of the ortholog (e.g., BBH) to the respective query gene; and (iii) whether the ortholog (e.g., BBH) of the respective query gene is co-localized with the ortholog (e.g., BBH) of the anchor gene (e.g., core synthase gene) in the respective genome; and b) inputting the grid representation into a machine-learning model, wherein the machine-learning model is trained to determine a likelihood that the putative embedded gene is embedded in the gene cluster (e.g., the BGC) based on values of the plurality of cells in the grid representation, thereby providing the likelihood that the putative embedded gene is associated with the gene cluster (e.g., the BGC). In some embodiments, the anchor gene is the core synthase gene (i.e., the longest biosynthetic gene) of the gene cluster. In some embodiments, an ortholog of a gene is a BBH of the gene. In some embodiments, the grid representation is a data matrix (e.g., a table). In some embodiments, the grid representation is a heat map. In some embodiments, the grid representation is a subset of a larger grid representation (e.g., a subsection of a larger data matrix or heat map). In some embodiments, the grid representation and/or one or more subsections thereof may be used as input for the machine learning model.

In some embodiments according to any one of the computer-based methods described above, the method further comprises creating the grid representation. In some embodiments, the method further comprises: a) identifying a putative gene cluster (e.g., a putative BGC) comprising a putative embedded gene in a query genome from a plurality of genomes, wherein the putative gene cluster comprises an anchor gene (e.g., core synthase gene) known to associate with the gene cluster, and wherein the anchor gene is co-localized with the putative embedded gene; b) identifying a plurality of positive genomes comprising an ortholog (e.g., BBH) of the anchor gene and a plurality of negative genomes that do not comprise an ortholog (e.g., BBH) of the anchor gene, wherein the plurality of positive genomes have pairwise sequence similarities of no more than a threshold value, and wherein the plurality of negative genomes are selected based on sequence similarities or phylogenetic distances to the plurality of positive genomes; and c) creating a grid representation comprising a plurality of cells arranged according to a first axis and a second axis, wherein the first axis corresponds to all protein-coding genes that are co-localized with the anchor gene in the putative gene cluster in the query genome, and the second axis corresponds to the plurality of positive genomes and the plurality of negative genomes, wherein each cell is based on: (1) the presence or absence of an ortholog (e.g., BBH) of the respective protein-coding gene in the respective genome; (2) sequence similarity of the ortholog (e.g., BBH) to the respective protein-coding gene; and (3) whether the ortholog of the respective protein-coding gene is co-localized with the ortholog of the anchor gene in the respective genome.

In some embodiments according to any one of the computer-based methods described above, the machine-learning model is a classification model configured to output a probability for each of a plurality of predefined likelihood categories (e.g., categories of likelihood that the putative embedded gene is embedded in the gene cluster (e.g., a BGC)). In some embodiments, the classification model is a long short-term memory (LSTM) model. In some embodiments, the classification model is a convolutional neural network (CNN) model. In some embodiments, the classification model is a vision transformer model, a generative adversarial network model, a variational autoencoder model, or a latent diffusion model. In some embodiments, for example, the plurality of predefined likelihood categories comprise: (1) high likelihood; (2) more likely than not; (3) more unlikely than not; and (4) low likelihood.

In some embodiments according to any one of the computer-based methods described above, the machine-learning model is a regression model configured to output a probability that the putative embedded gene is associated with the gene cluster (e.g., a BGC). In some embodiments, the regression model is a logistic regression model.

In some embodiments according to any one of the computer-based methods described above, the method further comprises displaying the grid representation and the likelihood.

In some embodiments according to any one of the computer-based methods described above, the grid representation is ordered along the first axis and/or the second axis. In some embodiments, the grid representation is hierarchically clustered, e.g., along the first axis or the second axis. In some embodiments, the grid representation is ordered along the first axis based on phylogenetic relationship among the plurality of genomes (e.g., based on a phylogenetic tree of the plurality of genomes). In some embodiments, the grid representation is ordered along the first axis based on pairwise sequence similarities among the plurality of genomes. In some embodiments, the grid representation is ordered along the second axis based on positions of the plurality of query genes in the query genome. In some embodiments, the grid representation is ordered along the second axis based on functional annotations of the plurality of query genes in the query genome.

In some embodiments according to any one of the computer-based methods described above, the plurality of genomes comprises a plurality of positive genomes each having an ortholog of the anchor gene (e.g., core synthase gene) and a plurality of negative genomes that do not have an ortholog of the anchor gene (e.g., core synthase gene). In some embodiments, the number of the positive genomes is equal to the number of negative genomes. In some embodiments, the plurality of positive genomes is selected from a plurality of genome clusters based on sequence similarities of genomes in a database, wherein no two positive genomes in the grid representation belong to the same genome cluster. In some embodiments, each negative genome is selected by identifying a genome in the database that has the highest sequence similarity or shortest phylogenetic distance to a positive genome, but does not have an ortholog of the anchor gene. In some embodiments, the average pairwise percentage sequence identities of orthologs of one or more single copy genes in the positive genomes are no more than about 99.5% (e.g., no more than about any one of 99%, 98%, 95%, 90%, 85%, 80%, 75%, 70%, 65%, 60%, 55%, or 50%), and/or the average pairwise percentage sequence identities of orthologs of one or more single copy genes in the negative genomes are no more than about 99.5% (e.g., no more than about any one of 99%, 98%, 95%, 90%, 85%, 80%, 75%, 70%, 65%, 60%, 55%, or 50%).

In some embodiments according to any one of the computer-based methods described above, the first axis corresponds to at least 2, 4, 8, 16, 20, 30, 40, 50, 75, 100, 150, 200, 250 or more genomes. In some embodiments, the first axis corresponds to about 50 genomes.

In some embodiments according to any one of the computer-based methods described above, the plurality of genomes are fungal genomes. In some embodiments, the plurality of genomes are Plantae (e.g., green algae and/or plant) genomes. In some embodiments, the plurality of genomes are bacterial genomes. In some embodiments, the plurality of genomes are archacal genomes. In some embodiments, the plurality of genomes are protozoan genomes. In some embodiments, the plurality of genomes are Chromista (e.g., brown algae, diatoms, crypophytes, etc.) genomes. In some embodiments, the plurality of genomes are Animalia genomes.

In some embodiments according to any one of the computer-based methods described above, whether a gene is co-localized with an anchor gene (e.g., core synthase gene) of a gene cluster (e.g., a BGC) is determined using antiSMASH, SMURF, TOUCAN, or deepBGC. In some embodiments, whether a gene is co-localized with an anchor gene (e.g., core synthase gene) of a gene cluster (e.g., a BGC) is determined using antiSMASH. In some embodiments, whether a gene is co-localized with an anchor gene (e.g., core synthase gene) of a gene cluster (e.g., a BGC) is determined based on whether the gene is located within a proximity zone upstream or downstream from the anchor gene (e.g., core synthase gene). In some embodiments, the proximity zone is no more than about any one of 200 kb, 100 kb, 90 kb, 80 kb, 70 kb, 60 kb, 50 kb, 45 kb, 40 kb, 35 kb, 30 kb, 25 kb, 20 kb, 15 kb, 10 kb or 5 kb. In some embodiments, the proximity zone is at least about any one of 5 kb, 10 kb, 15 kb, 20 kb, 25 kb, 30 kb, 35 kb, 40 kb, 45 kb, 50 kb, 60 kb, 70 kb, 80 kb, 90 kb, 100 kb or more. In some embodiments, the proximity zone is about any one of 5 kb-20 kb, 5 kb-50 kb, 5 kb-100 kb, 5 kb-200 kb, 20 kb-50 kb, 20 kb-100 kb, 20 kb-200 kb, 50 kb-100 kb, 50 kb-200 kb, 10 kb-50 kb, or 10 kb-100 kb. In some embodiments, the proximity zone is about 50 kb. In some embodiments, the proximity zone is about 20 kb.

Another aspect of the present application provides a method for identifying a resistance gene against a secondary metabolite produced by a BGC in a query genome, comprising: (a) identifying a putative embedded gene co-localized (e.g., within a proximity zone of no more than 50 kb, 20 kb, or any user-specified distance) with an anchor gene (e.g., core synthase gene) in the BGC in the query genome, wherein the putative embedded gene is not involved in the production of the secondary metabolite by the BGC; (b) performing the method according to any one of the computer-based methods described herein to determine the likelihood that the putative embedded gene is associated with the BGC; and (c) identifying the putative embedded gene as a resistance gene based at least in part on the likelihood that the embedded gene is associated with the BGC.

Another aspect of the present application provides a method for identifying a small molecule modulator of a mammalian target gene (i.e., a mammalian gene of interest), comprising: (a) identifying a homologous gene of the mammalian target gene in a fungal genome, wherein the homologous gene is co-localized with an anchor gene (e.g., core synthase gene) of a BGC of the fungal genome, and wherein the homologous gene is not involved in production of a secondary metabolite by the BGC; (b) performing the method according to any one of the computer-based methods described herein to determine the likelihood that the homologous gene is associated with the BGC; and (c) identifying the secondary metabolite, or an analog thereof, as a small molecule modulator of the mammalian target gene based at least in part on the likelihood that the homologous gene is associate with the BGC. In some embodiments, the homologous gene encodes a protein having at least about 20%, 25%, 30%, 35%, 40%, 45%, 50%, 55%, 60%, 65%, 70%, 85%, 90%, 95% or higher sequence identity or homology to a protein encoded by the mammalian target gene. In some embodiments, the homologous gene encodes a protein having at least about 30% sequence identity or homology to a protein encoded by the mammalian target gene. In some embodiments, the method further comprises contacting the secondary metabolite or analog thereof with a protein encoded by the mammalian target gene, and detecting an activity (e.g., binding to the secondary metabolite or analog thereof) of the protein encoded by the mammalian target gene.

Another aspect of the present application provides a method of identifying a plurality of genes associated with a BGC, comprising: (a) identifying a plurality of query genes that are co-localized with an anchor gene (e.g., core synthase gene) of a BGC in a query genome; (b) for each of the plurality of query genes, determining a likelihood that the respective query gene is associated with the BGC using the method according to any one of the computer-based methods described above; and (c) identifying query genes having a high likelihood of being associated with the BGC as the plurality of genes associated with the BGC, wherein the high likelihood is beyond a threshold value.

One aspect of the present application provides a computer-implemented method, comprising: a) identifying a putative biosynthetic gene cluster (BGC) comprising a putative embedded gene in a query genome from a plurality of genomes, wherein the putative BGC comprises an anchor gene known to associate with the BGC, and wherein the anchor gene is co-localized with the putative embedded gene; b) identifying a plurality of positive genomes comprising an ortholog of the anchor gene and a plurality of negative genomes that do not comprise an ortholog of the anchor gene, wherein the plurality of positive genomes have pairwise sequence similarities of no more than a threshold value, and wherein the plurality of negative genomes are selected based on sequence similarities or phylogenetic distances to the plurality of positive genomes; and c) creating a grid representation comprising a plurality of cells arranged according to a first axis and a second axis, wherein the first axis corresponds to all protein-coding genes that are co-localized with the anchor gene in the putative BGC in the query genome, and the second axis corresponds to the plurality of positive genomes and the plurality of negative genomes, wherein each cell is based on: (1) the presence or absence of an ortholog of the respective protein-coding gene in the respective genome; (2) sequence similarity of the ortholog to the respective protein-coding gene; and (3) whether the ortholog of the respective protein-coding gene is co-localized with the ortholog of the anchor gene in the respective genome. In some embodiments, the anchor gene is the core synthase gene (e.g., the longest biosynthetic gene) of the BGC. In some embodiments, an ortholog of a gene is a bidirectional best hit (BBH) of the gene. In some embodiments, the method further comprises hierarchically clustering the grid representation. In some embodiments, the method further comprises ordering the grid representation along the first axis, e.g., based on phylogenetic relationship (e.g., a phylogenetic tree) among the plurality of genomes, or based on pairwise sequence similarities (e.g., a cladogram derived from pairwise genome comparisons) among the plurality of genomes. In some embodiments, the grid representation is a data matrix (e.g., a table). In some embodiments, the grid representation is a heat map. In some embodiments, the method further comprises displaying the grid representation. In some embodiments, the grid representation is a subset of a larger grid representation (e.g., a subsection of a larger data matrix or heat map). In some embodiments, the grid representation and/or one or more subsections thereof may be used as input for the machine learning model.

Also provided is a non-transitory computer-readable storage medium that stores one or more programs, the one or more programs comprising instructions which, when executed by one or more processors of an electronic device, cause the electronic device to perform any of the techniques or methods, or one or more computer-based steps of the methods described herein.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE FIGURES

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 illustrates an exemplary putative biosynthetic gene cluster (BGC) as predicted by antiSMASH.

FIG. 2 illustrates an exemplary method for generating a grid representation (e.g., heat map) of genomic data.

FIG. 3 illustrates an exemplary method for determining a likelihood that a putative embedded gene co-localized with a core synthase gene of a gene cluster (e.g., a BGC) in a query genome is associated with the BGC, according to some examples.

FIGS. 4A-1 to 4A-3 illustrate an exemplary heat map.

FIGS. 4B-1 to 4C-2 illustrate an exemplary long short-term memory (LSTM) model used for classification of an input heat map into one of a plurality of likelihood categories (e.g., four likelihood categories or tiers). FIG. 4B-1 and FIG. 4B-2: exemplary illustration of an LSTM model comprising a series of memory tiers. FIG. 4C-1 and FIG. 4C-2: exemplary illustration of an output of an LSTM model.

FIG. 5A illustrates a table comparing manual classification versus machine-learning based classification of heat maps for “Tier A+”, “Tier 1”, “Tier 2”, and “Tier 3” categories, respectively.

FIG. 5B illustrates a table comparing manual classification versus machine-learning based classification of heat maps for “Tier A+”, “Tier 1”, “Tier 2”, and “Tier 3”, including a positive predictive value, a negative predictive value, a sensitivity value, and a specificity value.

FIGS. 6A-B illustrate an exemplary heat map of a BGC for lovastatin that identifies the true borders of the lovastatin BGC as compared to the predicted lovastatin BGC by antiSMASH.

FIGS. 7A-1 to 7D-2 illustrate exemplary heat maps manually reviewed and classified as different likelihood categories. FIG. 7A-1 and FIG. 7A-2: exemplary heat map classified as “Tier A+”. FIG. 7B-1 and FIG. 7B-2: exemplary heat map classified as “Tier 1”. FIG. 7C-1 and FIG. 7C-2: exemplary heat map classified as “Tier 2”. FIG. 7D-1 and FIG. 7D-2: exemplary heat map classified as “Tier 3”.

FIG. 8A illustrates a data table of a set of features (e.g., up to 27 features or more) that may be organized into a data table and utilized to train a neural network.

FIG. 8B illustrates an initial training phase of a neural network trained to output a probability value that a putative embedded gene (e.g., a pETaG) is associated with a BGC (i.e., an “embedded gene probability value” (e.g., an ETaG) probability value”)).

FIG. 8C illustrates a further training phase of a neural network trained to output an embedded gene probability value (e.g. an ETaG probability value).

FIG. 8D illustrates an inference phase of a neural network trained to output an ETaG probability value.

FIG. 8E illustrates an example data table including the unknown input data set of features and the corresponding outputs of ETaG probability values for each of the features.

### DETAILED DESCRIPTION

The present disclosure provides methods for identifying genes associated with a gene cluster (e.g., a biosynthetic gene cluster (BGC)) by comparative genomics analysis using a grid representation (e.g., a heat map). The grid representation enables visual and machine-learning based evaluation of co-occurrence and co-localization between genes found in proximity to a gene cluster (e.g., a BGC), biosynthetic or non-biosynthetic genes thereof, or other gene(s) of interest.

The most commonly used bioinformatics tool to identify biosynthetic gene clusters is antiSMASH, which annotates more than 40 types of BGCs based on the presence of certain key protein domains. Currently, there is no good method available to accurately predict the genomic boundaries of a gene cluster, e.g., a BGC. antiSMASH defines a BGC region using the following algorithm. In the first step, all gene products of the analyzed sequence are searched against a database of Hidden Markov model (HMM) profiles for highly conserved enzyme (e.g., core-enzymes), which are indicative of a specific BGC type. In a second step, pre-defined cluster rules are employed to define individual “Clusters” encoded in the analyzed sequence regions. Each identified cluster includes core gene-product(s), or core synthase gene(s), that trigger the cluster rule. antiSMASH defines a BGC region by extending to the upstream and downstream of the core synthase gene(s) by a predetermined length, such as 20 kb. The predetermined lengths for the different cluster types are empirically determined and generally tend to over-include adjacent genes as part of a BGC. See, e.g., Blink K et al., (2017) Nucleic Acids Res., 45, W36-W41; and Weber T. et al., antiSMASH5, antiSMASH Database Manual (2019). Therefore, genes identified as part of a BGC using antiSMASH, or based on proximity to a core synthase gene of a BGC, may not be functionally related to the secondary metabolite produced by the BGC.

To solve this problem, the methods and systems described herein leverage comparative genomics and machine-learning algorithms to assess heat maps representing a distribution of orthologs, e.g., bidirectional best hits (BBHs), of genes co-localized with an anchor gene (e.g., a core synthase gene) known to associate with a gene cluster (e.g., a BGC) across a number of diverse genomes in order to determine a likelihood that an ortholog of a query gene (e.g., a gene in a reference or query genome) that is co-localized with an anchor gene (e.g., a core synthase gene) of a gene cluster (e.g., a BGC) in the query genome is associated with the gene cluster (e.g., a BGC) in a target or queried genome. The machine-learning models can be trained using manually curated heat maps, including manually curated heat maps that represent co-localized non-biosynthetic genes that are known or experimentally verified to associate with gene clusters (such as BGCs), co-localized non-biosynthetic genes that are known or experimentally verified to have no functional association with gene clusters (such as BGCs,), and genes that comprise borderline cases. The trained machine-learning algorithms allow rapid assessment of a large number of putative embedded genes that are co-localized with anchor genes (e.g., core synthase genes) in gene clusters such as BGCs using sequence information from a large number of genomes, which greatly improves the accuracy of delineating the borders of the gene cluster. Furthermore, the methods allow prioritization of putative embedded genes in gene clusters such as BGCs for downstream assessment, including assessment through time-consuming and costly experimental verification processes.

The methods described herein can be used to define gene cluster boundaries, i.e., to identify functionally related genes that are co-localized on a chromosome. The gene clusters, or protein products thereof, may participate in a variety of cellular functions, such as biosynthesis (e.g., secondary and primary metabolism), immunity, cell structure, scavenging, energy and sensing. In particular, the methods described herein can be used to define borders of a BGC in different genomes by identifying genes that are associated with the BGC.

Furthermore, the methods can be used to identify a resistance gene (e.g., a gene that confers resistance to the action of the secondary metabolite produced by a BGC to the host organism) which is embedded in the BGC required for the production of the secondary metabolite. The identification of BGC embedded resistance genes enables the de-orphanization of the BGC-encoded small molecule's protein target (ETaG product), which may have a homolog in a mammalian genome. The mammalian homologs of the ETaG may serve as candidate therapeutic targets, and the secondary metabolite can provide small molecule scaffolds for developing modulators against such mammalian homologs.

### Definitions

As used herein, a “secondary metabolite” refers to an organic small molecule compound produced by archaca, bacteria, fungi or plants, which are not directly involved in the normal growth, development, or reproduction of the host organism, but are required for interaction of the host organism with its environment. Secondary metabolites are also known as natural products or genetically encoded small molecules. The term “secondary metabolite” is used interchangeably herein with “biosynthetic product” when referring to the product of a biosynthetic gene cluster.

The terms “biosynthetic gene cluster” or “BGC” are used herein interchangeably to refer to a locally clustered group of one or more genes that together encode a biosynthetic pathway for the production of a secondary metabolite. Exemplary BGCs include, but are not limited to, biosynthetic gene clusters for the synthesis of non-ribosomal peptide synthetases (NRPS), polyketide synthases (PKS), terpenes, and bacteriocins. See, for example, Keller N, “Fungal secondary metabolism: regulation, function and drug discovery.” Nature Reviews Microbiology 17.3 (2019): 167-180 and Fischbach M. and Voigt C. A., PROKARYOTIC GENE CLUSTERS: A RICH TOOLBOX FOR SYNTHETIC BIOLOGY. In: Institute of Medicine (US) Forum on Microbial Threats. The Science and Applications of Synthetic and Systems Biology: Workshop Summary. Washington (DC): National Academies Press (US); 2011. A21. BGCs contain genes encoding signature biosynthetic proteins that are characteristic of each type of BGC. The longest biosynthetic gene in a BGC is referred to herein as the “core synthase gene” of a BGC. In addition to genes involved in the biosynthesis of a secondary metabolite, a BGC may also include non-biosynthetic genes, i.e., genes that encode products that are not involved in the biosynthesis of a secondary metabolite, which are interspersed among the biosynthetic genes. The non-biosynthetic genes are referred herein as being “associated” or “embedded” with the BGC if their products are functionally related to the secondary metabolite of the BGC. An “anchor gene” refers to a biosynthetic or non-biosynthetic gene that is co-localized with a BGC and is known to be functionally related (i.e., associated) with the BGC.

The term “co-localize” refers to the presence of two or more genes in close spatial proximity, such as genes separated by no more than about 200 kb, no more than about 100 kb, no more than about 50 kb, no more than about 40 kb, no more than about 30 kb, no more than about 20 kb, no more than about 10 kb, no more than about 5 kb, or less, in a genome.

The term “homolog” refers to a gene that is part of a set of genes wherein the gene sequences (i.e., nucleic acid sequences) and/or the sequences of their protein products are inherited through a common origin. Homologs may arise through speciation events, or through gene duplication events, or through horizontal gene transfer events. Homologs may be identified by phylogenetic methods, or through identification of common functional domains in the aligned nucleic acid or protein sequences, or through sequence comparisons.

The term “orthologs” refers to two or more genes that are predicted to have evolved from a common ancestral gene by speciation. The terms “bidirectional best hit” and “BBH” are used herein interchangeably to refer to the relationship between a pair of genes in two genomes, i.e., a first gene in a first genome and a second gene is a second genome, wherein the first gene or its protein product has been identified as having the most similar sequence in the first genome as compared to the second gene or its protein product in the second genome, and wherein the second gene or its protein product has been identified as having the most similar sequence in the second genome as compared to the first gene or its protein product in the first genome. The first gene is the bidirectional best hit (or BBH) of the second gene, and the second gene is the bidirectional best hit (or BBH) of the first gene. Identification of BBHs is a commonly used method to infer orthology.

“Percent (%) sequence identity” or “percent (%) sequence homology” with respect to the protein sequences described herein is defined as the percentage of amino acid residues in a candidate polypeptide sequence that are identical or homologous with the amino acid residues in the polypeptide to which it is being compared, after aligning the sequences and considering any conservative substitutions as part of the sequence identity. Homology between different amino acid residues is determined based on a substitution matrix, such as the BLOSUM (BLOcks SUbstitution Matrix). Alignment for the purposes of determining percent amino acid sequence identity can be achieved in various ways known to those of skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

As used herein, “sequence similarity” between two genes means similarity of either the nucleic acid (e.g., DNA or mRNA) sequences encoded by the genes or the amino acid sequences of the gene products.

In the following description, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating” or the like, refer to the action and processes of a computer system, or similar electronic computing device, that manipulates and transforms data represented as physical (electronic) quantities within the computer system memories or registers or other such information storage, transmission, or display devices.

The section headings used herein are for organizational purposes only and are not to be construed as limiting the subject matter described. The description is presented to enable one of ordinary skill in the art to make and use the invention, and is provided in the context of a patent application and its requirements.

### Grid Representation Analysis Methods

The systems and methods described herein relate to identification of genes associated with a gene cluster, e.g., a biosynthetic gene cluster (BGC), using machine-learning algorithms to assess grid representations (e.g., heat maps, or data matrices) of orthologs of genes that co-localize with an anchor gene (e.g., a core synthase gene) of the, e.g., BGC across diverse genomes.

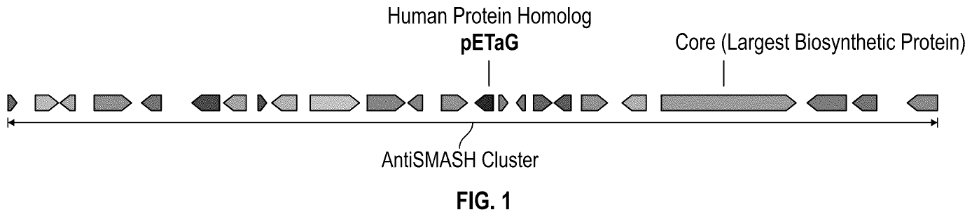

FIG. 1 illustrates an exemplary putative BGC region as predicted by antiSMASH. The BGC includes a series of genes that encode enzymes, including signature biosynthetic enzymes, the longest of which is referred herein as a “core biosynthetic protein”, in a biosynthetic pathway for the production of a secondary metabolite. Non-biosynthetic genes co-localized with biosynthetic genes in a BGC may be homologs of human proteins, including therapeutic targets of interests. Such non-biosynthetic genes may be functionally related to the secondary metabolite produced by the BGC, or encode functionally irrelevant products. A non-biosynthetic gene in a BGC, which is a homolog of a human protein of interest, is a putative embedded target gene (pETaG). The methods described herein leverage comparative genomics to determine the likelihood that a gene of interest is associated with a BGC.

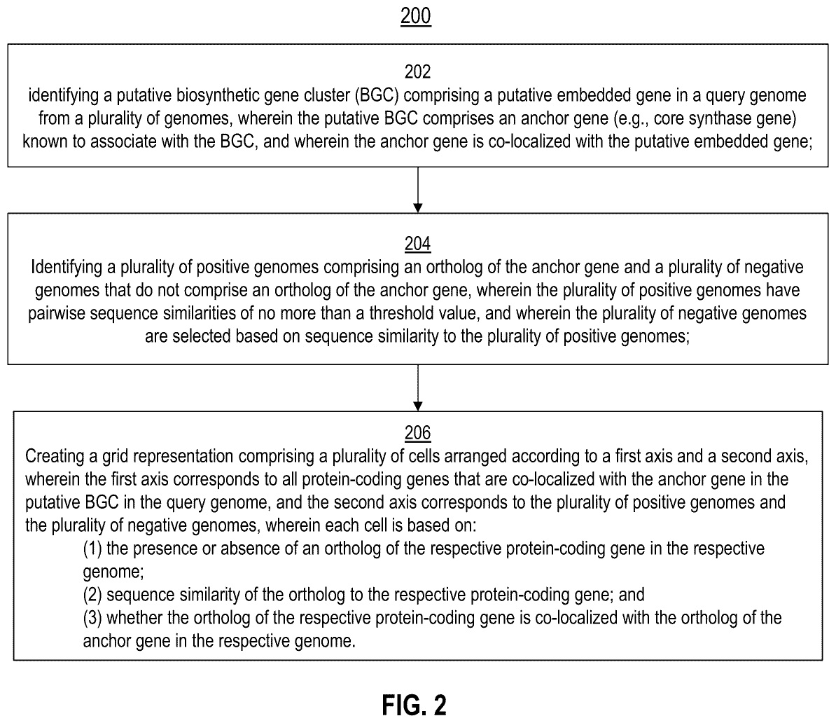

FIG. 2 illustrates an exemplary method 200 for generating a grid representation (e.g., a heat map) of genomic data that can be input into a machine-learning algorithm (such as an artificial neural network (ANN), a convolutional neural network (CNN), a multilayer perceptron (MLP), a deep neural network (DNN), an LSTM, a vision transformer model, a generative adversarial network (GAN) model, a variational autoencoder model, a latent diffusion model, etc.) to determine a likelihood that a pETaG is associated with a gene cluster (e.g., a BGC) in a genome.

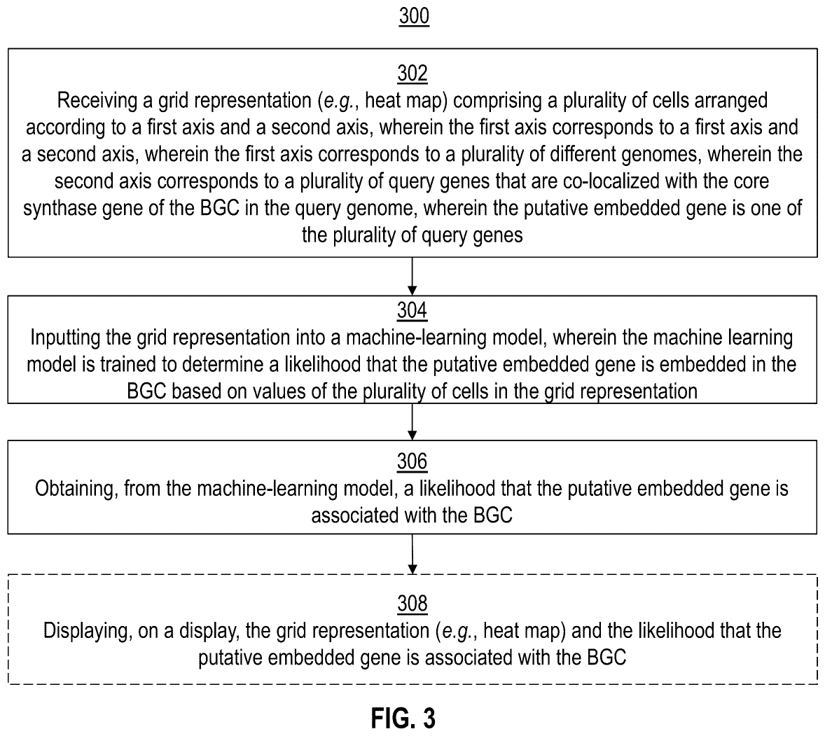

FIG. 3 illustrates an exemplary method 300 for determining a likelihood that a putative embedded gene is associated with a gene cluster (e.g., a BGC). Process 200 and process 300 are performed, for example, using one or more electronic devices implementing a software platform. In some examples, process 200 and/or process 300 is performed using a client-server system, and the blocks of process 200 and/or process 300 are divided up in any manner between the server and one or more client devices. In some examples, process 200 and/or process 300 is performed using only a client device or only multiple client devices. In process 200 and/or 300, some blocks are, optionally, combined, the order of some blocks is, optionally, changed, and some blocks are, optionally, omitted. In some examples, additional steps may be performed in combination with the process 200 and/or process 300. Accordingly, the operations as illustrated (and described in greater detail below) are exemplary by nature and, as such, should not be viewed as limiting.

### Grid Representation

At block 302 of FIG. 3, an exemplary system (e.g., one or more electronic devices) receives a grid representation of genomic data (such as a heat map representation) comprising a plurality of cells arranged according to a first axis and a second axis, wherein the first axis corresponds to a plurality of different genomes (e.g., non-mammalian genomes), wherein the second axis corresponds to a plurality of query genes that are co-localized with the anchor gene (e.g., a core synthase gene) of a gene cluster (e.g., a BGC) in the query (or reference) genome, wherein the putative embedded gene is one of the plurality of query genes. Each cell in the grid representation has a value based on the following: (i) whether an ortholog of the respective query gene (i.e., the query gene corresponding to the cell) is present or absent in the respective genome (i.e., the genome corresponding to the cell); (ii) sequence similarity of the ortholog to the respective query gene; and (iii) whether the ortholog of the respective query gene is co-localized with the ortholog of the anchor gene (e.g., core synthase gene) in the respective genome.

The grid representation described herein can take any of a variety of forms known to those of skill in the art. For example, the grid representation may be a data matrix, such as a two-dimensional data matrix (e.g., a table or array) arranged according to the first axis and the second axis and having the values described herein for each of the cells in the data matrix. In some embodiments, the grid representation comprises one or more matrices (e.g., tables) of data. For example, each set of values (i.e., (i) whether an ortholog of the respective query gene (i.e., the query gene corresponding to the cell) is present or absent in the respective genome (i.e., the genome corresponding to the cell); (ii) sequence similarity of the ortholog to the respective query gene; and (iii) whether the ortholog of the respective query gene is co-localized with the ortholog of the anchor gene (e.g., core synthase gene) in the respective genome) of the cells in the grid representation, or combinations thereof, may be stored in a separate table and used as an input for the machine learning-based methods described herein. In some embodiments, the grid representation may be a physical representation of the underlying data matrix, for example, a heat map, which facilitates visualization of the data.

With respect to each query gene in the query (or reference) genome, an ortholog in any given genome (e.g., a target genome) may be identified based on the coding sequence of the query gene or the protein sequence encoded by the query gene, or based on phylogenetic relationships using known methods in the art. For example, an ortholog of a query gene in a given genome may be a gene in the given genome that encodes a protein having the highest sequence similarity to the query gene, or for which the sequence similarity is above a pre-determined threshold. Sequence similarities can be quantified by any of a variety of parameters known to those of skill in the art, including percent sequence identity, percent sequence homology, bitscore, and e-value. The pre-determined threshold may be a percent sequence identity or percent sequence homology of, for example, at least about any one of 20%, 30%, 40%, 50%, 60%, 70%, 80%, 85%, 90%, 95%, 96%, 97%, 98%, 99% or higher.

In some embodiments, an ortholog of a query gene in a given genome may be a bidirectional best hit (BBH) of the query gene in the given genome. The method for identifying a BBH has been described, for example, in Moreno-Hagelsieb G, Latimer, K., Bioinformatics. 2008 Feb. 1; 24 (3): 319-24. For example, to identify a BBH of a query gene in a given genome, the given genome is first searched for the gene (“putative BBH”) encoding a protein having the highest sequence similarity to the protein encoded by the query gene. This search is followed by a reciprocal search, in which the query genome is searched for the gene encoding a protein having the highest sequence similarity to the putative BBH identified in the query genome. If the gene identified in the reciprocal search is the original query gene, the putative BBH is a true BBH. Alternatively, an ortholog of a query gene in a given genome may be identified using a reciprocal smallest distance method, e.g., as described in Wall D P, Deluca T, Methods Mol Biol. 2007; 396:95-110.

The query genes, including the putative embedded genes, in a query (or reference) genome are co-localized with an anchor gene (e.g., core synthase gene) of a gene cluster, e.g., a BGC. Two genes may be considered co-localized if one gene is within a specified distance, or proximity zone, of the other. In some instances, the distance between two genes can be considered, for example, the shortest distance between the genomic coordinates of the two genes. For example, if gene A resides on the + strand and comprises a sequence ranging from positions 1 to 100, and gene B resides on the − strand and comprises a sequence ranging from positions 300 to 200 (i.e., where position 300 is the start of gene sequence B due to its position on the − strand), then the distance between the two genes is 200−100=100 bp. In some instances, the distance between two genes can be considered the longest distance between the genomic coordinates of the two genes. In some instances, the distance between the two genes can be considered the distance between the midpoint genomic coordinates for the two genes. A putative embedded gene in a target genome is co-localized with an anchor gene (e.g., core synthase gene) of a BGC in the target genome when the putative embedded gene is within a specified proximity zone relative to the anchor gene (e.g., core synthase gene) in the BGC of the target genome. In some embodiments, a proximity zone is about 1-100 kb, for example, no more than about 1, 2, 3, 4, 5, 6, 7, 8, 9, 10, 15, 20, 30, 40, 50, 60, 70, 80, 90, or 100 kb upstream or downstream of an anchor gene (e.g., core synthase gene) in a BGC. In some embodiments, a proximity zone is about 1-10 kb, for example, no more than 1, 2, 3, 4, 5, 6, 7, 8, 9, or 10 kb upstream or downstream of an anchor gene (e.g., core synthase gene) in a gene cluster (e.g., a BGC). In some embodiments, a proximity zone is no more than 5 kb upstream or downstream of a gene. In some embodiments, a proximity zone is no more than 10 kb upstream or downstream of a gene. In some embodiments, a proximity zone is no more than 15 kb upstream or downstream of a gene. In some embodiments, a proximity zone is no more than 20 kb upstream or downstream of a gene. In some embodiments, a proximity zone is no more than 25 kb upstream or downstream of a gene. In some embodiments, a proximity zone is no more than 30 kb upstream or downstream of a gene. In some embodiments, a proximity zone is no more than 35 kb upstream or downstream of a gene. In some embodiments, a proximity zone is no more than 40 kb upstream or downstream of a gene. In some embodiments, a proximity zone is no more than 45 kb upstream or downstream of a gene. In some embodiments, a proximity zone is no more than 50 kb upstream or downstream of a gene.

A putative BGC, for example, includes both biosynthetic genes and non-biosynthetic genes, and may further include pseudogenes (e.g., nonfunctional segments of DNA that resemble functional genes). Curated libraries of profile Hidden Markov Model (pHMMs) of biosynthetic domains of BGCs (i.e., probabilistic models that capture the biological diversity of the biosynthetic domains based on a position-dependent scoring of multiple sequence alignments for the corresponding protein or nucleic acid sequences) are known in the art and can be used to identify biosynthetic genes that are clustered in a genome. An anchor gene may be any one of the signature genes associated with a BGC. For example, an anchor gene may be the core synthase gene in a BGC, which is the largest biosynthetic gene in a BGC, or the anchor gene may be the core synthase gene closest to a query gene. Alternatively, an anchor gene may be a non-biosynthetic gene that is known to associate with a BGC, for example, a gene encoding a transporter for the secondary metabolite produced by a BGC. In some embodiments, the plurality of genes in a putative BGC may be identified by extending a window of predetermined length upstream and downstream from an anchor gene (e.g., core synthase gene). In some embodiments, the plurality of genes in a putative BGC of a given genome can be identified using bioinformatics methods, such as antiSMASH.

For example, a grid representation may have a first axis (e.g., Y-axis) corresponding to genomes Q₁ to Qₙ and a second axis (e.g., X-axis) corresponding to genes G₁ to Gₘ. A cell corresponding to genome Qᵢ (1≤i≤n) and gene Gj (1≤i≤n) has a value and a color selected from a first color, a second color and a third color according to the following:
(i) if Qᵢ does not have a BBH in Gⱼ, the color is the first color and the value is 0; or
(ii) if Qᵢ has a BBH in Gⱼ, the value is based on percentage sequence identity of Gⱼ to the BBH of Gⱼ in Qᵢ, and
   (ii-1) if the BBH in Gⱼ is co-localized with the BBH of the core synthase gene in Qᵢ, the color is the second color, or
   (ii-2) if the BBH in Gⱼ is not co-localized with the BBH of the core synthase gene in Qᵢ, the color is the third color.

The grid representation may be hierarchically clustered in order to assist visualization and manual annotation. For example, the grid representation by be clustered based on the pairwise sequence identity or homology among the genomes, phylogeny of the genomes, or the presence or absence of orthologs corresponding to all query genes in the grid representation. In some embodiments, the first axis of the grid representation (e.g., heat map) is organized according to a phylogenetic tree of the plurality of genomes represented in the grid representation.

In some embodiments, a putative embedded gene is a putative embedded target gene (pETaG), which encodes a homolog of a mammalian protein of interest, such as a homolog of a human protein of interest. In some embodiments, a pETaG is homologous to an expressed mammalian nucleic acid sequence. In some embodiments, a mammalian nucleic acid sequence is an expressed mammalian nucleic sequence. In some embodiments, a mammalian nucleic acid sequence is a mammalian gene. In some embodiments, a mammalian nucleic acid sequence is an expressed mammalian gene. In some embodiments, a mammalian nucleic acid is a human nucleic acid sequence. In some embodiments, a human nucleic acid sequence is an expressed human nucleic acid sequence. In some embodiments, a human nucleic acid sequence is a human gene. In some embodiments, a human nucleic acid sequence is an expressed human gene.

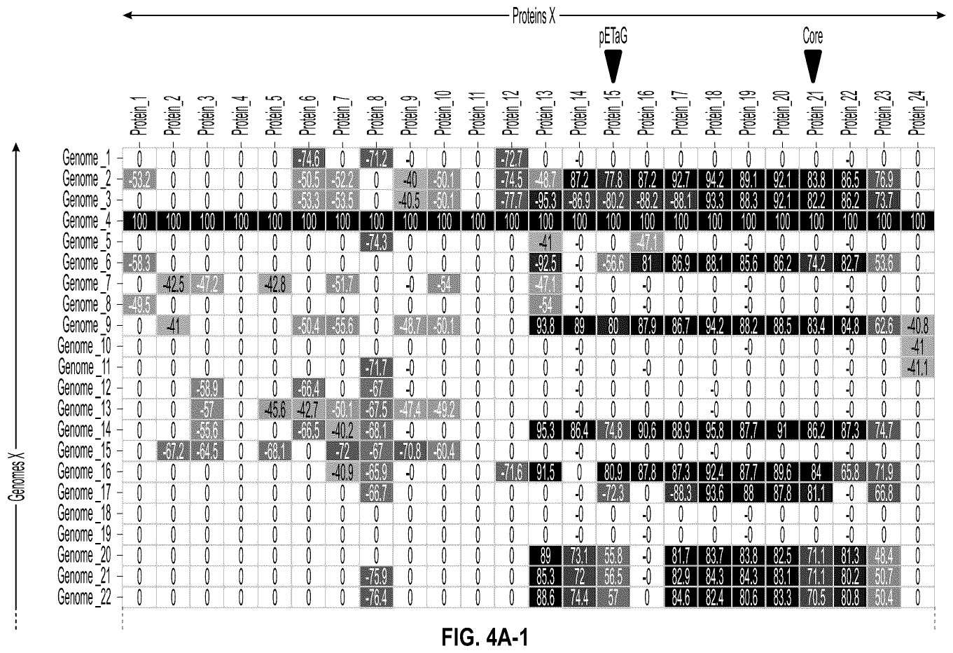

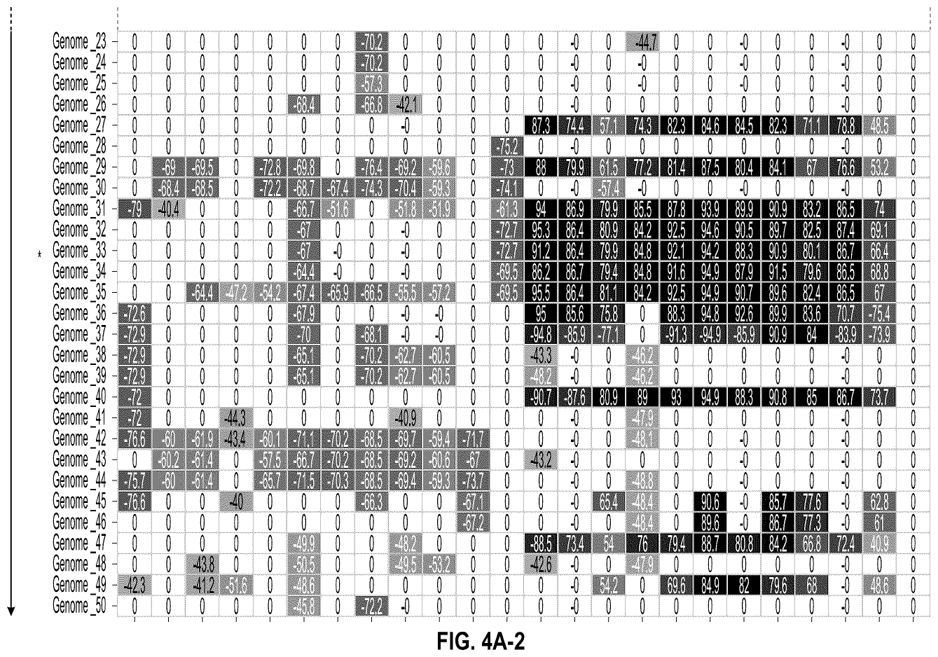

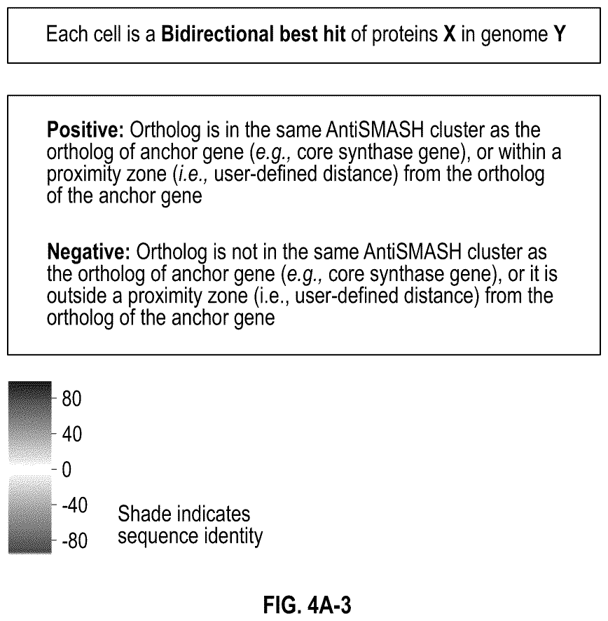

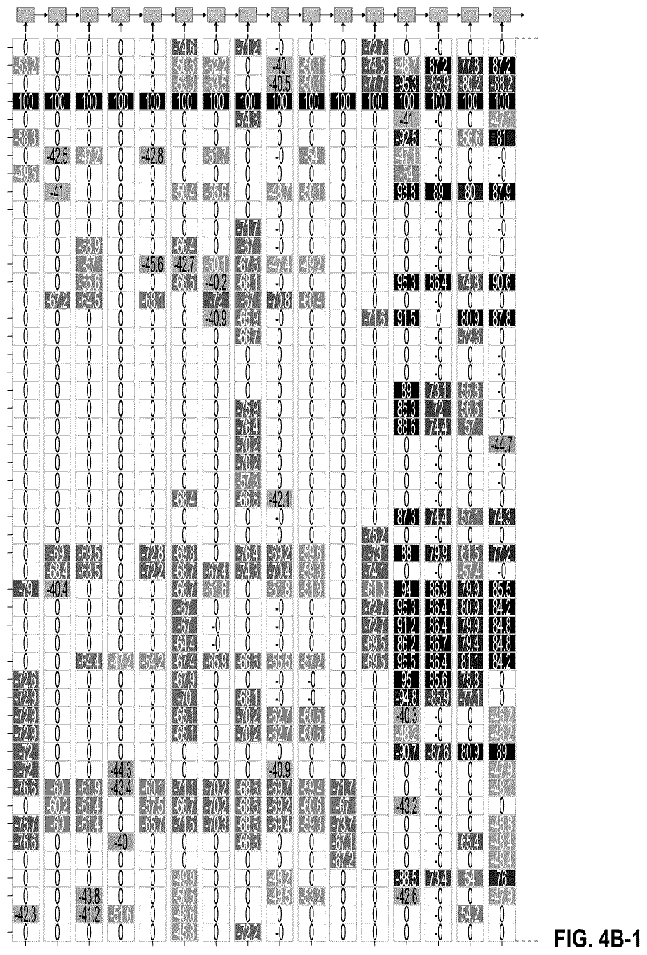

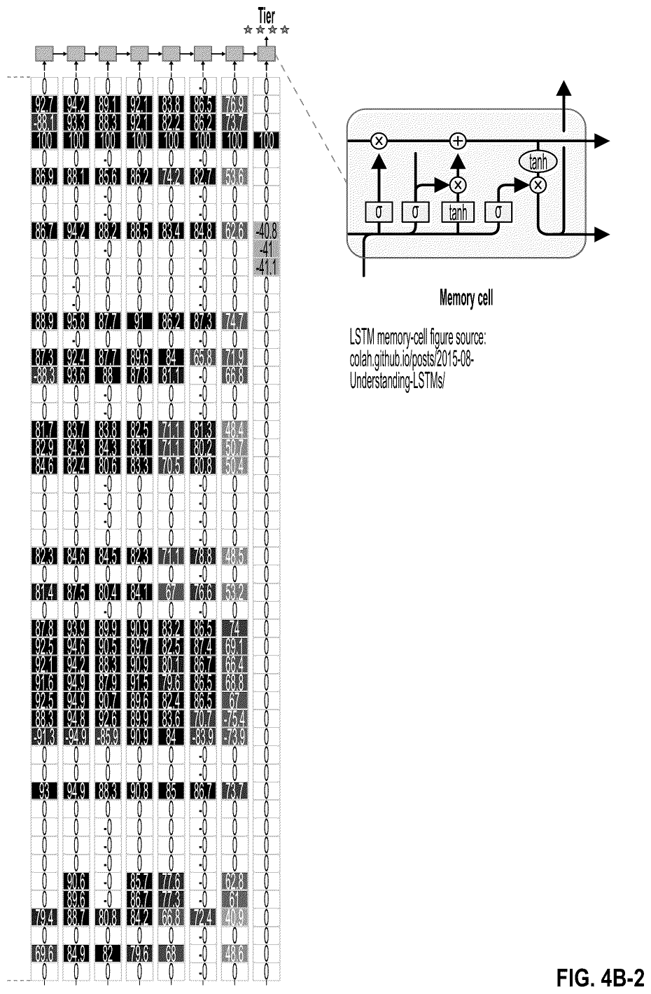

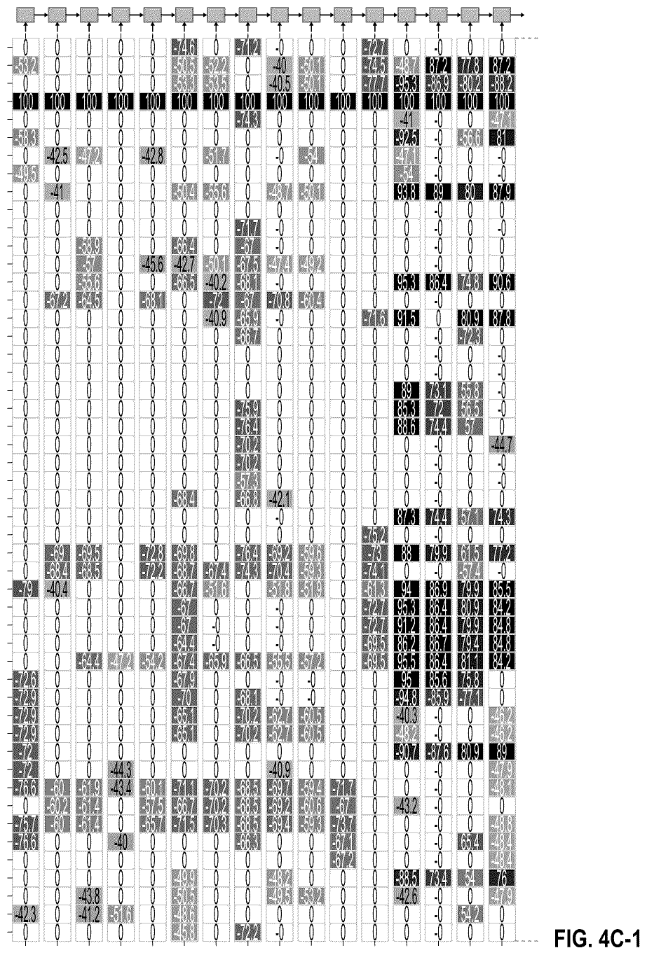

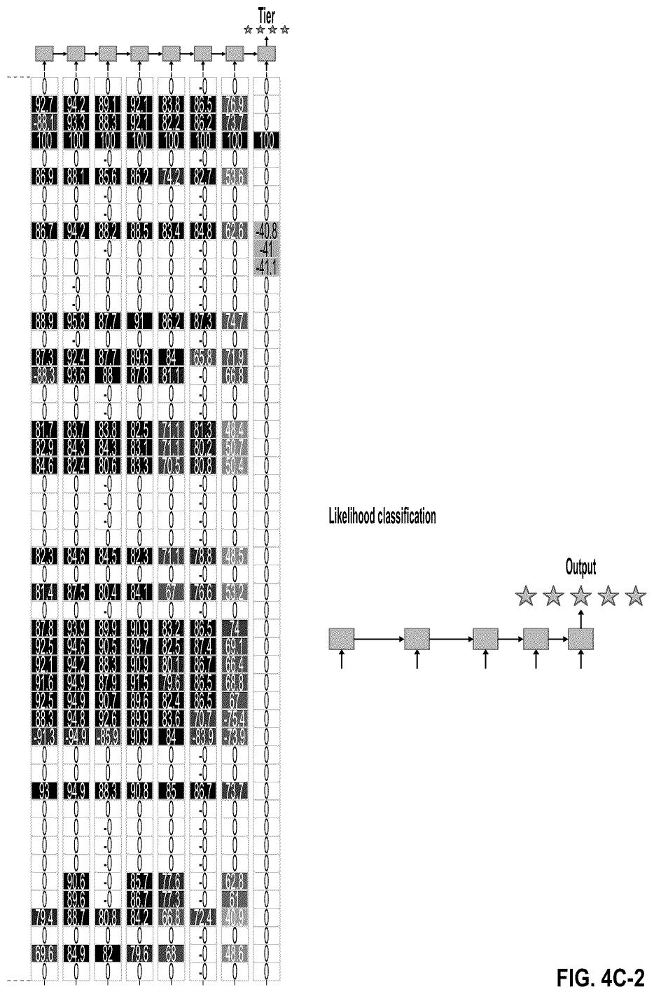

An example of a genomic heat map is shown in FIGS. 4A-1 to 4A-3. This example illustrates a heatmap for a pETaG in a BGC identified by antiSMASH in the genome marked by an asterisk (*). Each of the columns along the X-axis represents a protein (“Protein X”) encoded by a query gene in the BGC identified in the query (or reference) genome marked by an asterisk (*). In some embodiments, the BGC is identified by antiSMASH. In some embodiments, the genes in the BGC are within a proximity zone of 20 kb from the core synthase gene of the BGC (i.e., within +20 kb of the core synthase gene). The columns corresponding to a pETaG and the core synthase gene are marked with arrows. Each of the rows along the Y-axis represents a unique genome (“Genome Y”) selected from the genome database. Half of the genomes contain a BBH of the core synthase gene, and are designated positive genomes. Half of the genomes do not contain a BBH of the core synthase gene, and are designated as negative genomes. Each cell is colored or shaded according to the presence or absence of a BBH of the respective query gene, and the percentage sequence identity (numbers in cells) of the BBH to the respective query gene. For example, cell (X,Y) is blank if there is no BBH of Protein X in Genome Y; cell (X, Y) is, e.g., blue or positive, if there is a BBH of Protein X in Genome Y and the BBH is in the same antiSMASH BGC cluster as the BBH of the core synthase gene in Genome Y; or cell (X, Y) is e.g., red or negative, if there is a BBH of Protein X in Genome Y and the BBH is not in the same antiSMASH BGC cluster as the BBH of the core synthase gene in Genome Y. The intensity of the red or blue color (or grayscale shading) of cell (X, Y) is based on percentage sequence identity of the BBH of Protein X in Genome Y to Protein X. The heat map is hierarchically clustered based on pairwise sequence identities among the genomes.

The genomes shown in the grid representation may each correspond to an assembled genome, or a plurality of genome fragments obtained from genome sequencing. In some embodiments, the genomes are annotated using bioinformatics tools, such as antiSMASH, prior to the analysis using any one of the methods described herein. For example, a database of genomes can be constructed so that all putative biosynthetic gene clusters have been identified and annotated. For example, genome fragments containing the putative BGCs, instead of full genomes, may be queried in one or more steps of the methods described herein. For example, co-localization of genes (e.g., orthologs of query genes) with an anchor gene (e.g., core synthase gene) can be determined based on the putative BGC annotations in the genomes.

The methods described herein are suitable for any genomes containing BGCs. Bacterial genomes, plant genomes and fungal genomes are known to encode biosynthetic gene clusters. In some embodiments, the query (or reference) genome and the plurality of queried (or target) genomes used to generate the grid representation belong to the same kingdom. In some embodiments, the query genome and the plurality of queried genomes used to generate the grid representation belong to different kingdoms. Suitable genomes include, but are not limited to genomes of Archaca, Protozoa, Chromista (e.g., brown algae, diatoms, crypophytes, etc.), Plantac (e.g., green algae and plants), Fungi, and Animalia. In some embodiments, the query genome and the plurality of queried genomes are fungal genomes, such as genomes of different fungal strains. In some embodiments, the query genome and the plurality of queried genomes are bacterial genomes, such as genomes of different bacterial strains. In some embodiments, the query genome and the plurality of queried genomes are plant genomes, such as genomes of different plant strains. Without wishing to be bound by any theory or hypothesis, fungal genomes are eukaryotic genomes that are phylogenetically more related to mammalian genomes than bacterial or plant genomes. Thus, fungal genomes may be preferred for identification of ETaGs that correspond to human target genes (i.e., human genes of interest) for the secondary metabolites produced by the BGCs harboring the ETaGs.

At least 2 genomes are required for building the grid representation. In some embodiments, the first axis of the grid representation corresponds to at least about any one of 10, 15, 20, 25, 30, 35, 40, 45, 50, 60, 70, 80, 90, 100, 150, 200, 250 or more genomes. In some embodiments, the first axis of the grid representation corresponds to at least 20 genomes. In some embodiments, the first axis of the grid representation corresponds to about 50 genomes. Although a large number of genomes may provide more comparative genomic information, it also requires a large amount of computational power and time. Thus, it may be desirable to sample a representative set of genomes that are diverse with respect to each other in terms of their sequence similarities and/or phylogenetic relationships to generate the grid representation to strike a balance between performance of the methods (e.g., accuracy of prediction) and computational resource.

The genomes shown in the grid representation may include “positive genomes” and “negative genomes.” Positive genomes are those genomes having orthologs of the anchor gene, such as the core synthase gene, in the query (or reference) genome. Negative genomes are those genomes that do not have orthologs of the anchor gene, such as the core synthase gene, in the query (or reference) genome. In some embodiments, the positive and negative genomes are selected from a database of genomes. In some embodiments, the plurality of genomes used to build the grid representation comprises a plurality of positive genomes each having an ortholog (e.g., BBH) of the anchor gene (e.g., core synthase gene), and a plurality of negative genomes that do not have an ortholog (e.g., BBH) of the anchor gene (e.g., core synthase gene). In some embodiments, the positive genomes are selected from a genome database by identifying genomes having an ortholog (e.g., BBH) of the anchor gene (e.g., core synthase gene), and the negative genomes are selected form genome database by identifying genomes without an ortholog (e.g., BBH) for the anchor gene (e.g., core synthase gene). Negative genomes may be phylogenetically adjacent to the selected positive genomes. In some embodiments, the number of positive genomes and the number of negative genomes are equal to each other.

The positive and negative genomes may be selected from a database having a large number of genomes. For example, a database may contain at least 2, 10, 100, 500, 1000, 5000, 10000, 15000, 20000, 25000, 30000, 35000, 40000, 45000, 50000, 100000, 200000, 500000, 1000000, or more than 1000000 genomes. Selection of the positive and negative genomes from a large genome database may require clustering of the genomes in order to allow sampling of diverse genomes, including positive and negative genomes, from the database. For example, in some instances the database genomes may be clustered according to the sequence similarities of one or more single copy genes in the genomes, or the sequence similarities of orthologs of the anchor gene (e.g., core synthase gene), or the putative embedded gene (e.g., a pETaG) in the genomes. The clustering may be performed using an unsupervised clustering method. The unsupervised clustering method may comprise the use of, for example, a Markov Cluster algorithm (MCL), a Restricted Neighborhood Search Cluster (RNSC) algorithm, an Affinity Propagation clustering algorithm, a Spectral clustering algorithm, a k-means clustering algorithm, or any other method known in the art. The clustering may alternatively comprise the use of a supervised clustering method that is known in the art, such as supervised k-means clustering or semi-supervised spectral clustering. The threshold for clustering may be determined by a predetermined goal for the number of clusters. For example, the threshold for clustering may be a pre-determined sequence similarity level among the group of genomes, e.g., requiring sequence similarities between different groups of genomes to be no more than about any one of 99.5%, 99%, 98%, 95%, 90%, 85%, 80%, 75%, 70%, 65%, 60%, 50%, 40%, 30%, or less. In some embodiments, it may be desirable to select positive genomes in which the pairwise percent sequence similarities (e.g., sequence identity) of orthologs of one or more single copy genes in the positive genomes to be more than about any one of 99.5%, 99%, 98%, 97%, 96%, 95%, 94%, 93%, 92%, 91%, 90%, 85%, 80%, 70%, 60%, 50%, 40%, 30%, or less. In some embodiments, it may be desirable to select negative genomes in which the pairwise percent sequence similarities (e.g., sequence identity) of orthologs of one or more single copy genes in the negative genomes to be more than about any one of 99.5%, 99%, 98%, 97%, 96%, 95%, 94%, 93%, 92%, 91%, 90%, 85%, 80%, 70%, 60%, 50%, 40%, 30%, or less. A representative genome from each of the clusters can be further selected for use in the analytical steps as described herein. In some embodiments, a negative genome is selected from a database by identifying the genome having the highest sequence similarity to a positive genome, but that lacks an ortholog of the anchor gene (e.g., core synthase gene).

For example, a grid representation may be built based on a number, n, of positive genomes selected from a database of m genomes. As a first step, a number of positive genomes, m₁, having an ortholog (e.g., BBH) of the anchor gene (e.g., core synthase gene) and a number, (m−m₁), of negative genomes without an ortholog (e.g., BBH) of the anchor gene (e.g., core synthase gene) are identified from the m number of genomes in the database. In the situation where my is larger than n, the m₁ positive genomes are clustered using MCL based on the average sequence similarities of one or more single copy genes (e.g., identified using the BUSCO tool, see, busco.czlab.org) among the positive genomes into n clusters. One positive genome is then selected from each of the n clusters to provide n positive genomes for building the grid representation. Each of the n negative genomes is selected by identifying the most similar genome (e.g., the genome with the highest sequence similarity to, or with the shortest phylogenetic distance from) a selected positive genome among the (m−m₁) negative genomes. This genome selection method results in a grid representation built from n positive genomes and n negative genomes.

In the situation where my is smaller than n, then more negative genomes may be selected than positive genomes in order to build a grid representation having a total of 2n genomes. In this case, the (m−m₁) negative genomes may be clustered into (2n−m₁) clusters, and one negative genome is chosen from each cluster. Alternatively, for each of the m₁ positive genomes, more than one negative genome that is closely related to the positive genome is chosen based on their sequence similarities or phylogenetic distances to the positive genome, so that a total of 2n−m₁ negative genomes are selected.

The grid representation may be generated, for example, using the method illustrated in FIG. 2.

As a first optional step, resource files can be prepared for the downstream computational processes. Exemplary resource files include a pairwise genome comparison file, a file (e.g., FASTA file) containing relevant proteins or genes from target genomes (i.e., genomes selected for the analysis), and optionally, a resource file containing Clusters of Orthologous Groups of proteins or genes (COGs).

A pairwise genome comparison file is a file created to show the homology relationship between all pairs of genomes in a database. Genome similarities can be determined based on either pairwise genome sequence similarities, or pairwise phylogenetic distances between genomes.

In some examples, pairwise identities or similarities among genomes can be determined by either comparing whole genome sequences, or by comparing sequences of subsets of proteins or genes. For example, to determine whole genome sequence identities, the entire genomes can be aligned and the pairwise identities between the alignments are computed. Alternatively, pairwise genome identities can be calculated by comparing single copy proteins (i.e., amino acid sequences) or genes (i.e., nucleotide sequences) shared between the pair of genomes. In some preferred embodiments, single copy proteins or genes are used as duplicated or fragmented proteins may provide misleading estimates of genome homology. Single copy proteins or genes in a genome can be annotated using BUSCO (doi.org/10.1093/molbev/msab199), or specific known single-copy proteins or genes can be used to determine genome sequence similarities. In some embodiments, single-copy proteins can be identified using known bioinformatic tools such as OrthoMCL, OrthoFinder, or PanX. A subset or all single copy proteins or genes shared between the genomes are individually aligned, trimmed, and concatenated to create a super-alignment. The pairwise identity is the number of identical residues in the super-alignment. Alternatively, a similarity score can be calculated based on substitution matrices, such as BLOSUM and PAM if protein sequences are used to determine sequence similarities.

In other examples, genome similarities are determined based on phylogenetic distances between the genomes. To determine phylogenetic distances, a set of single-copy proteins (i.e., amino acid sequences) or genes (i.e., nucleotide sequences) can be individually aligned using any alignment software such as MAFFT, MUSCLE, or ClustalW, trimmed using any sequence trimming software such as trimAI, GBlocks, or ClipKIT, and concatenated to create a super-alignment. The super-alignment can be used by any phylogenetic tree building software such as FastTree, IQ-TREE, RAXML, MEGA, MrBayes, BEAST, or PAUP to provide a phylogenetic tree of the genomes. Trees can be constructed using different algorithms, such as maximum likelihood algorithms, parsimony algorithms, neighbor joining algorithms, distance matrix algorithms, or Bayesian inference algorithms. Alternatively, a gene-coalescent phylogenetic model approach can be used to re-construct the phylogeny instead of the super-alignment approach.

In some examples, a FASTA file containing all protein or gene sequences from target genomes is created as an input resource file. Alternatively, a smaller subset of proteins, such as proteins in putative gene clusters (e.g., a putative BGCs) based on bioinformatics predictions, can be supplied as an input instead of all proteins from all genomes. The putative BGCs, for example, can be predicted using publicly available BGC prediction tools such as antiSMASH, SMURF, TOUCAN, deepBGC, or using custom BGC prediction tools. The FASTA file may contain either protein sequences or nucleic acid sequences as sequence similarities (e.g., homology) may be determined using either protein sequences or nucleic acid sequences.

Clusters of Orthologous Groups (COG) of gene cluster (e.g., BGC) proteins or genes may be provided as an optional resource file. Members of the same COG are presumed to have orthologous functions. COGs can be created using protein clustering tools such as USEARCH, CD-HIT, and MMseqs. Alternatively, COGs can be generated using clustering algorithms such as MCL after performing sequence (e.g., amino acid or nucleotide) alignment searches with BLAST, ggsearch, or Diamond. COGS can also be identified using custom developed scripts or using known bioinformatics tools such as OrthoMCL, OrthoFinder, or PanX. COGs can also be defined as gene families or protein families. The COG file may, for example, contain protein or gene IDs from the same COG in each row of a table.

At block 202 of FIG. 2, the method for generating a grid representation comprises identifying a putative gene cluster (e.g., a putative biosynthetic gene cluster (BGC)) comprising a putative embedded gene a query (or reference) genome from a plurality of genomes, wherein the putative gene cluster comprises an anchor gene (e.g., core synthase gene) known to associate with the gene cluster (e.g., the BGC), and wherein the anchor gene is co-localized with the putative embedded gene. In some examples, the method comprises identifying the longest biosynthetic or structural gene in the putative gene cluster as the anchor gene. The resource files can be used to perform the step of block 202. This step establishes the first axis of a grid representation, e.g., the X-axis of a heat map, which corresponds to a plurality of query genes that are co-localized with the putative gene cluster (e.g., BGC) in the query genome and that comprises the putative embedded gene. Co-localization may be determined either based on putative BGC annotations, or based on the distance between two genes, e.g., two genes within a specified proximity zone of no more than about 50 kb, or no more than about 20 kb.

For example, the X-axis of a heat map can be established based on a single protein or gene ID of interest (e.g., a gene ID corresponding to a pETaG), or based on multiple protein or gene IDs of interest as input. If multiple protein or gene IDs are input, then correlation and co-localization of these genes (along with flanking genes) with respect to each other across multiple genomes are determined. For example, the multiple protein or gene IDs of interest may correspond with an ETaG and a core synthase gene in a query (or reference) genome. If a single protein or gene ID is used as an input, then one can determine if the flanking genes surrounding it are co-localized across multiple genomes. The X-axis corresponds to the search region that contains the flanking genes of the protein or gene(s) (e.g., pETaG) of interest. The search region can be defined through either a predicted BGC or based on a coordinate location on the genomes. Putative BGCs can be predicted using tools such as antiSMASH, SMURF (dx.doi.org/10.1016/j.fgb.2010.06.003), TOUCAN (doi.org/10.1093/nargab/lqaa098), deepBGC (doi.org/10.1093/nar/gkz654) or other custom search algorithms.

If a predefined region is used as an input, then it is assumed that the specified input protein(s) or corresponding gene ID(s) reside within the region. Alternatively, a custom flanking distance (i.e., a proximity zone distance) specified in units of, e.g., base pairs (bp) can be used to identify a genome window region that comprises a certain number of bps upstream and downstream flanking either side of the specified protein or gene ID(s). All proteins (or genes) that are located within the defined region are assigned as X-axis proteins (or genes). The proteins (or genes) within the region are labeled. If the input protein ID is a single protein, the input label is used as the label for the protein ID. For example, one can input a core synthase gene and determine the genes that are co-localized with the core synthase.

If the input protein ID is a single protein and it is desired to determine its correlation with another gene (for which the ID was not input), then another specified input can be used to identify genes within the region to label as such. For example, if a gene of interest corresponding to an ETaG is used as an input and it is desired to determine if it is correlated with a core synthase, one can identify a search for core synthases and label identified proteins as such. Proteins can be searched based on gene annotations. If there are multiple target proteins within the region that match the requested search criteria, several options can be utilized. For example, heatmaps for each of the target proteins can be created. A target protein can be selected based on length or proximity of the target protein to the input protein ID. If multiple protein or gene IDs are used as an input, then the protein IDs are labeled based on their input labels. For example, one input protein can be labeled as the ETaG and the other input protein labeled as a core synthase.

At block 204 of FIG. 2, the method comprises obtaining a plurality of positive genomes comprising an ortholog of the core synthase gene and a plurality of negative genomes that do not comprise an ortholog of the core synthase gene, wherein the plurality of negative genomes are selected based on sequence similarities or phylogenetic distances to the plurality of positive genomes. The resource files can be used to perform the step of block 204. This step establishes the second axis of the grid representation, e.g., Y-axis of a heat map, which corresponds to the target genomes, including a plurality of positive genomes and a plurality of negative genomes.

For example, to establish the Y-axis of a heat map, positive and negative genome IDs can be obtained as follows. To establish positive genome IDs, protein homologs of the core synthase ID are searched for by running protein sequence alignment tools such as ggsearch, BLASTp, or Diamond-blastp against a set of genomes. Alternatively, genes (i.e., nucleotide sequences) can be used to find homologs for the gene of the core synthase in the set of genomes using tools such as ggsearch or BLASTn. Protein IDs falling within a specified range of minimum and maximum sequence identity are identified. A specified number of protein homologs from those having the highest sequence identity to the query core synthase may be selected. Core synthase homologs can be de-replicated by selecting representatives from protein clusters created by using protein clustering tools with a specified cutoff. Alternatively, de-replication can also be done using a BUSCO pairwise cutoff, phylogenetic distance cutoff, or taxonomic classifications. If a phylogenetic tree is used as a genome homology resource file, it can be traversed to select a diverse set of positive genomes that pass the criteria for presence of protein homologs. Alternatively, positive genomes can be selected based on pairwise BUSCO identity cutoffs, phylogenetic distance or clades, or taxonomic classifications (e.g., one isolate per species or one species per genera or family from genomes with protein homologs). These methods can be utilized to help ensure selection of a diverse set of genomes, which provides greater accuracy in identifying co-localized genes and avoids confounding results with multiple genomes of the same species, which can bias the results towards co-localization and increase the false positive rate. Each genome ID from the selected core synthase homologs is obtained and assigned as a positive genome.

To obtain negative genome IDs, the genome ID with the highest sequence identity or the closest phylogenetic distance to each positive genome may be selected using the pairwise genome homology file as an input. If a genome contains core synthase homologs within a specified range of sequence identity, the genome is dropped from the list of candidates and the search skips to the next candidate genome. If a phylogenetic tree is used as the homology resource file, then the tree can be traversed to find the closest negative genome to each positive genome. The positive and negative genome IDs are combined and assigned as the Y-axis genome IDs.

Optionally, files containing all protein (or nucleotide) sequences and gene annotation files (GFF, GTF, GenBank, or similar) related to Y-axis genomes are obtained. If gene cluster (e.g., BGC) predictions are used to define the search region, then gene cluster (e.g., BGC) information for the Y-axis genomes including gene cluster (e.g., BGC) IDs, cluster numbers, and protein (or gene) IDs located in the gene cluster (e.g., BGC) is stored as a resource file.

In some examples, a linkage matrix (also known as a cladogram) is built to visually show distances between all genomes of the Y-axis. The linkage matrix can be created using the pairwise homology matrix of the Y-axis genomes from the genome homology resource file (e.g., pairwise identities, similarities, or phylogenetic distance). A hierarchical clustering method can be used with the pairwise homology matrix to create a linkage matrix. Alternatively, the presence/absence of BBHs or forward alignment results of the X-axis protein homologs can be used with a hierarchical clustering method to create a linkage matrix. Alternatively, a phylogenetic tree can be used. The phylogenetic tree can be created from either a set of proteins (i.e., amino acid sequences) or transcripts (i.e., nucleotide sequences).

At block 206 of FIG. 2, the method comprises creating a grid representation comprising a plurality of cells arranged according to a first axis and a second axis, wherein the first axis corresponds to all protein-coding genes (e.g., query genes) that are co-localized with the anchor gene in the putative gene cluster (e.g., a putative BGC) in the query (or reference) genome, and the second axis corresponds to the plurality of positive genomes and the plurality of negative genomes, wherein each cell is based on: (1) the presence or absence of an ortholog of the respective protein-coding gene in the respective genome; (2) sequence similarity of the ortholog to the respective protein-coding gene; and (3) whether the ortholog of the respective protein-coding gene is co-localized with the ortholog of the anchor gene in the respective genome.

For example, the following steps can be used to create a heat map matrix. First, bi-directional best hits (BBH) results for X-axis proteins (or genes) in all Y-axis genomes are obtained to provide a BBH table. BBH is identified as a pair of proteins (or genes) from two different genomes that are more similar to each other than either is to any other gene in the other genome. BBH can be useful in identifying the true ortholog of a given protein (or gene), which is particularly helpful in identifying the current ortholog of genes that have had duplication events (such as ETaGs). Identification of BBHs involves a forward alignment step and a backward alignment step. In the forward alignment step, a sequence alignment tool is run using each X-axis protein as a query against the protein FASTA file of each Y-axis genome with specified cutoffs. Sequence alignment tools such as ggsearch, BLASTp, or Diamond-blastp can be used. Alternatively, genes (i.e., nucleotide sequences) can be used instead of protein sequences for the forward alignment step using tools such as ggsearch or BLASTn. The protein (or gene) ID of the best match from each alignment is stored in a table with X-axis proteins (or genes) as columns and Y-axis genomes as indices. The sequence identity of the best match from each alignment is stored in a table with X-axis proteins as columns and Y-axis genomes as indices. In the backward alignment step, a backward alignment is run using protein sequence alignment tools such as ggsearch, BLASTp, or Diamond-blastp. Each protein stored as the best hit from the forward alignment step is used as a query protein (or query gene). Protein alignment is performed against the protein FASTA file of the Y-axis genomes. Alternatively, genes (i.e., nucleotide sequences) can be used instead of protein sequences for the backward alignment using tools such as ggsearch or BLASTn. If the best hit from the backward alignment is the same as the query protein used in the forward alignment, then an X-axis protein (or gene) and its forward alignment hit are BBHs, and the BBH value in the table is defined as True. If the best hit from the backward alignment is different from the query protein used in the forward alignment, then an X-axis protein (or gene) and its forward alignment hit are not BBHs, and the BBH value in the table is defined as False. This binary data is stored in the BBH table with X-axis proteins as columns and Y-axis genomes as indices. Alternatively, only the forward alignment results are used instead of full BBH results.

In addition, a co-localization table is created. If, for example, BGC predictions are used to define the search region, then the cluster number is obtained, and the cluster number of each forward alignment hit from a table containing the BGC information of each genome is obtained. The values are stored in a table with X-axis proteins as columns and Y-axis genomes as indices. If the cluster number of each forward alignment hit is the same as the core synthase homolog of the genome, then the forward alignment hit is co-localized with the core synthase homolog, and the co-localization value is defined as True. Otherwise, the forward alignment hit is not co-localized with the core synthase homolog, and the co-localization value is defined as False. This binary information of co-localization is stored in the co-localization table.

Alternatively, if a custom flanking distance (i.e., proximity zone distance) is used to define the search region, then the genome location is obtained, and a table is created, which contains genome location of each forward alignment hit. The scaffold ID and the coordinates of the start and end positions of each protein are stored. A co-localization table that contains the binary information of the co-location between each forward alignment hit and a core synthase homolog of the corresponding genome is created. Co-localization value is defined as True if the query protein and the core synthase homolog are located within the specified distance (i.e., proximity zone) in the same scaffold.

A final table storing values for the cells of the heat map are created based on the BBH and co-localization tables as described above. The following transformations are applied: convert True to 1 and False to 0 from the binary data of the table containing BBH information; and convert True to 1 and False to −1 from the binary data of the table containing the co-localization information. To compute the value of each cell of the heat map, multiply sequence identity, BBH information, and the co-localization information value of all combinations with X-axis proteins and Y-axis genomes. For example, a cell may have the value of: Sequence identity (96.28)*BBH (1 or 0)*co-localization (1 or −1). If there is no BBH, the value of the cell becomes 0. If the cell corresponds to a gene not co-localized with the core synthase gene, the cell value becomes minus of the sequence identity. For the heat map based on forward alignment hits, the value of each cell is computed as the product of sequence identity and the co-localization information value of all combinations with X-axis proteins and Y-axis genomes. A heat map is plotted using the final table and the linkage matrix. A diverging colormap may be used to visualize the value from −100 to 100. The Y-axis can be reordered based on hierarchical clustering of the linkage matrix.

The generated grid representation and/or subsets of the generate grid representation (e.g., heat map, or data matrix) can be input into a machine-learning model, such as an LSTM, to provide a likelihood that the putative embedded gene (e.g., an pETaG) is associated with the BGC.

### Machine-Learning Model for Predicting “Embeddedness”

At block 304 of FIG. 3, the system inputs the grid representation into a machine-learning model, in which the machine-learning model is trained to determine a likelihood that the putative embedded gene is associated with the gene cluster (e.g., a BGC) based on values of the plurality of cells in the grid representation. Non-limiting examples of machine learning models that may be used include, but are not limited to, artificial neural networks (ANNs), convolutional neural networks (CNNs) or other recurrent neural networks (RNNs), multilayer perceptrons (MLPs), deep neural networks (DNNs), LSTMs, vision transformer models, generative adversarial networks (GANs), variational autoencoders, latent diffusion models, etc.

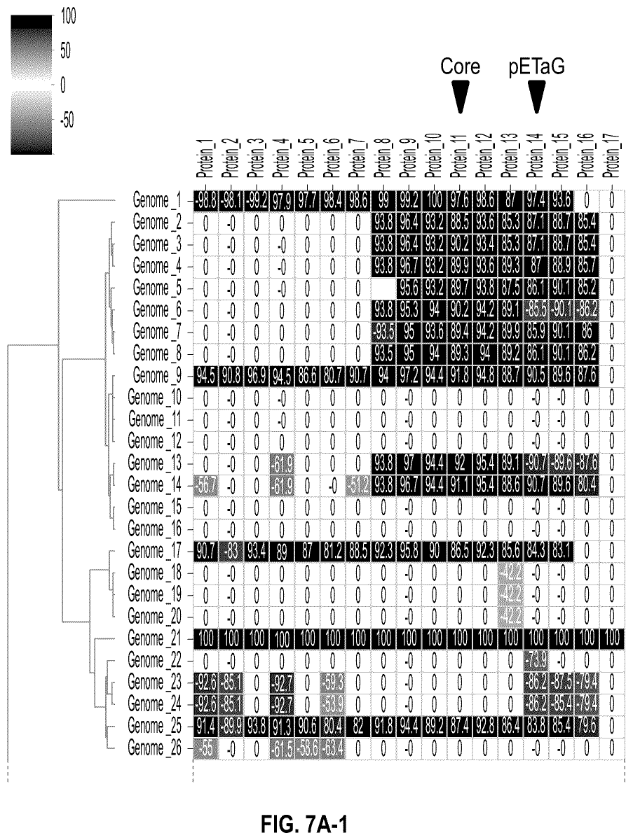

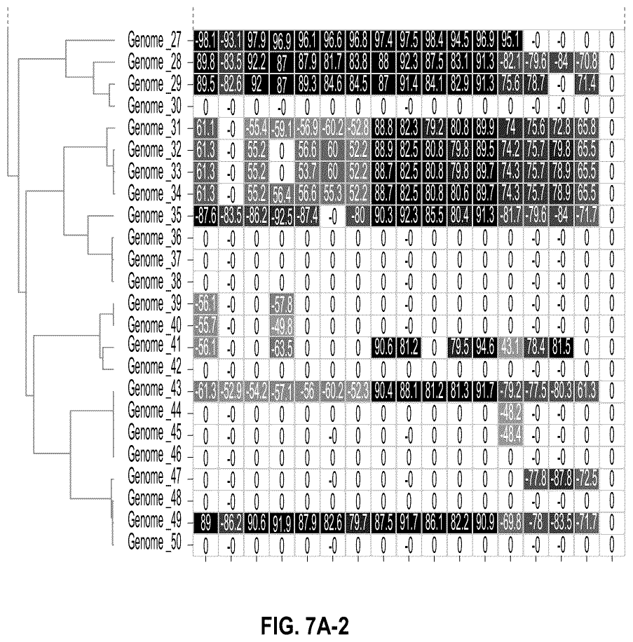

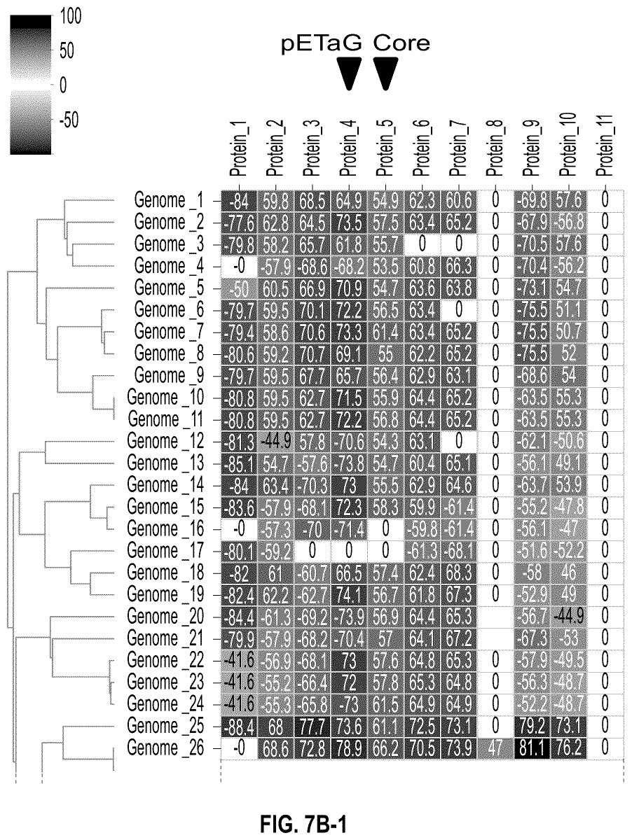

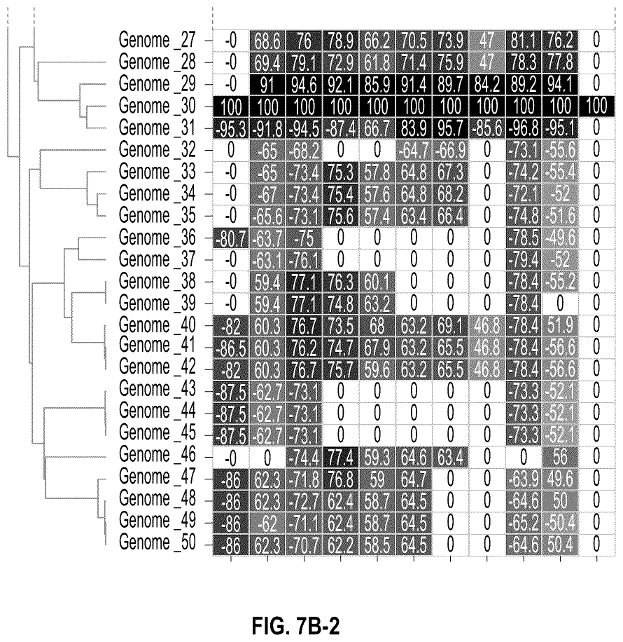

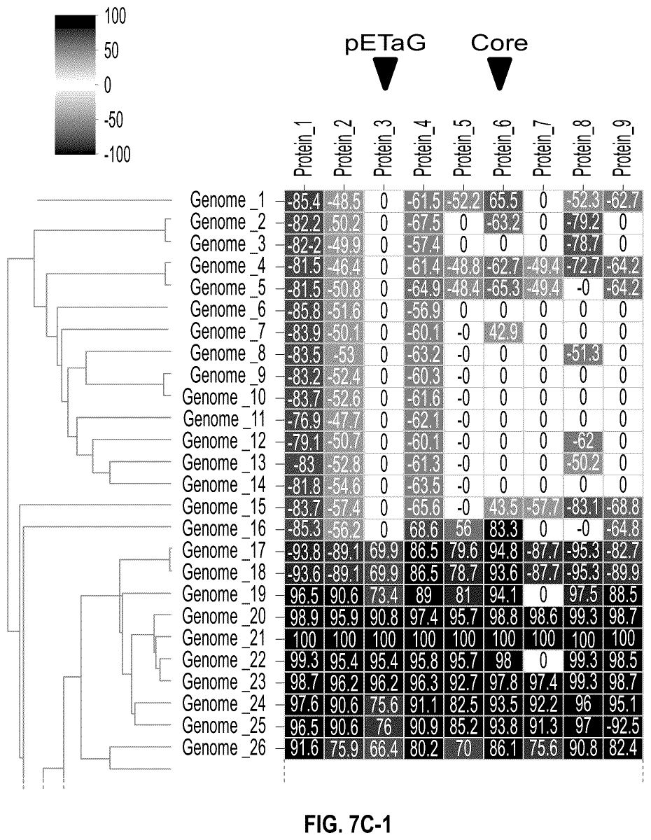

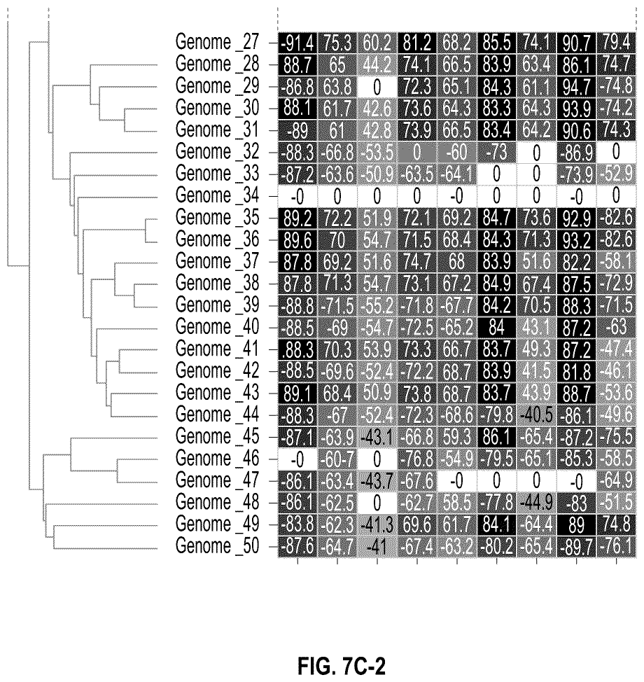

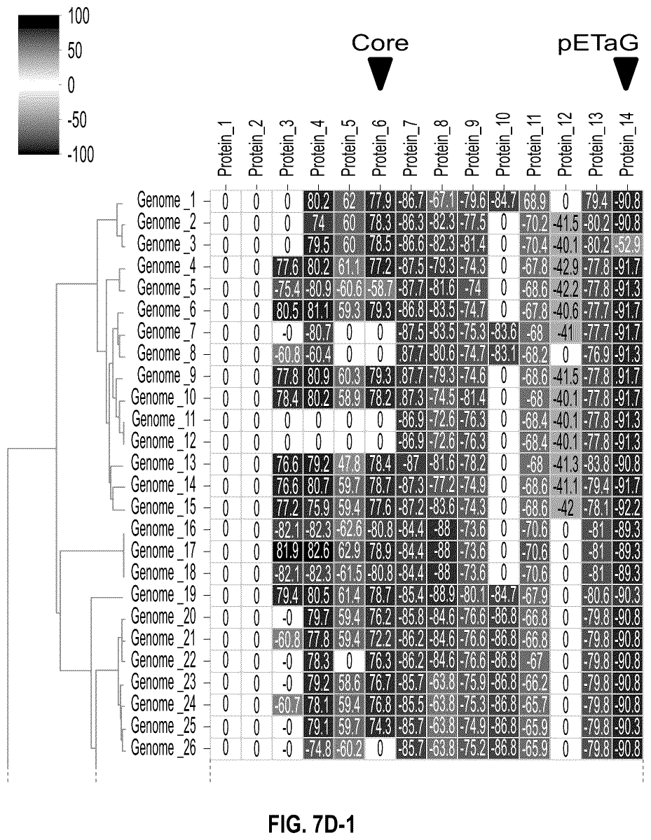

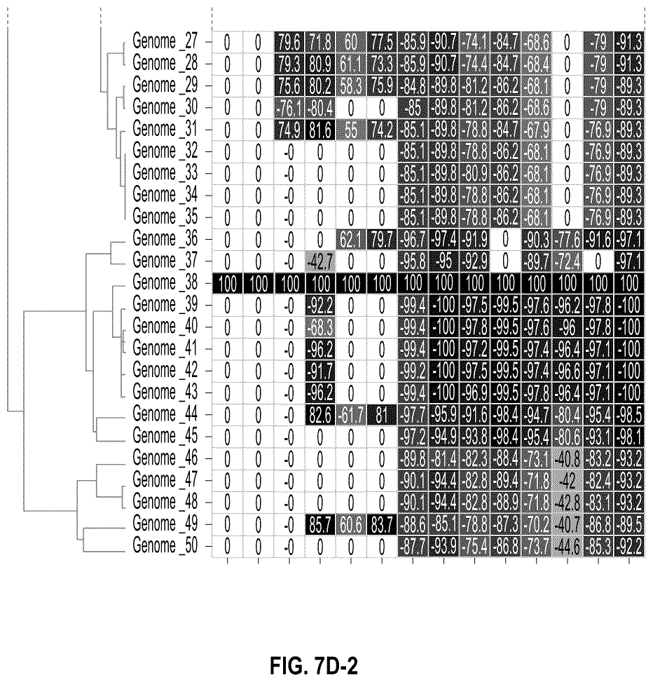

The likelihood may be a probability that the putative embedded gene is associated with the gene cluster (e.g., a BGC), or a probability that the putative embedded gene falls within one of a plurality of predefined likelihood categories. In some embodiments, the likelihood may fall within one of the following four categories: (1) high likelihood that the putative embedded gene is associated with the gene cluster (e.g., BGC) (also referred herein as “Tier A+”); (2) more likely than not that the putative embedded gene is associated with the gene cluster (e.g., BGC) (also referred herein as “Tier 1”); (3) more unlikely than not that the putative embedded gene is associated with the gene cluster (e.g., BGC) (also referred herein as “Tier 2”); and (4) low likelihood that the putative embedded gene is associated with the gene cluster (e.g., BGC) (also referred herein as “Tier 3”). Tier A+ heat maps have clearly defined gene cluster (e.g., BGC) boundaries, and the putative embedded gene (e.g., a putative resistance gene or pETaG) is within those boundaries. See, for example FIGS. 7A-1 to 7A-2. Tier 1 heat maps have gene cluster (e.g., BGC) boundaries that are not clearly defined, but the putative embedded gene (e.g., a putative resistance gene or pETaG) is correlated with the anchor gene (e.g., a core synthase gene). See, for example FIGS. 7B-1 to 7B-2. Tier 2 heat maps tend to provide insufficient information for identification of the gene cluster (e.g., BGC) boundaries, or allow acceptance or rejection of the correlation between the putative embedded gene (e.g., a putative resistance gene or pETaG) and the anchor gene (e.g., a core synthase gene). See, for example FIGS. 7C-1 to 7C-2. Tier 3 heat maps indicate that the putative embedded gene is a false positive because either: (1) the boundaries of the gene cluster (e.g., BGC) are clearly defined and the putative embedded gene (e.g., a putative resistance gene or pETaG) is not within the boundaries; or (2) there is no correlation or co-localization between the putative embedded gene (e.g., a putative resistance gene or pETaG) and the anchor gene (e.g., a core synthase gene). See, for example FIGS. 7D-1 to 7D-2. The method may output a probability associated with each of these likelihood categories. For example, the method may provide a probability associated with each of the four categories, in which the sum of the four probabilities is equal to 100%.

At block 306 of FIG. 3, the system obtains from the machine-learning model a likelihood that the putative embedded gene is associate with the gene cluster (e.g., BGC).

At block 308 of FIG. 3, the system displays, on a display, the grid representation (e.g., a heat map) and the likelihood that the putative embedded gene is associated with the gene cluster (e.g., BGC).

FIGS. 4A-1 to 4A-3 illustrate a heat map that represents the degree of “embeddedness” (i.e., association with a BGC) of a pETaG in the computed heat map, in accordance with the presently disclosed embodiments. In the heat map, the putative BGC is identified by antiSMASH in the genome marked by an asterisk (*). Each of the columns along the X-axis represents a protein (“Protein X”) encoded by a query gene in the BGC identified in the query genome marked by an asterisk (*). The genes in a BGC may be identified by antiSMASH or based on proximity (e.g., within 20 kb) to the core synthase gene of a BGC. The columns corresponding to the pETaG and the core synthase gene are marked with arrows. Each of the rows along the Y-axis represents a unique genome (“Genome Y”) selected from the genome database. Half of the genomes contain a BBH of the pETaG, and are referred herein as “positive genomes.” Half of the genomes do not contain a BBH of the pETaG, and are referred herein as “negative genomes.” Each cell is colored or shaded according to the presence or absence of a BBH of the respective query gene, and the percentage sequence identity (numbers in cells) of the BBH to the respective query gene. For example, cell (X, Y) is blank if there is no BBH of Protein X in Genome Y; cell (X,Y) is, e.g., blue or positive, if there is a BBH of Protein X in Genome Y and the BBH is in the same antiSMASH BGC cluster as the BBH of the core synthase gene in Genome Y; or cell (X, Y) is e.g., red or negative, if there is a BBH of Protein X in Genome Y and the BBH is not in the same antiSMASH BGC cluster as the BBH of the core synthase gene in Genome Y or if there is no BBH of the core synthase gene in Genome Y. The intensity of the red or blue color (or grayscale shading) of cell (X, Y) is based on percentage sequence identity of the BBH of Protein X in Genome Y to Protein X. The heat map is hierarchically clustered based on pairwise sequence identities among the genomes.

FIGS. 4B-1 to 4C-2 illustrate an exemplary long short-term memory (LSTM) model used for classification of an input heat map into one of four tiers.: (1) high likelihood that a putative embedded target gene (pETaG) is associated with the gene cluster (e.g., BGC) (“Tier A+”); (2) more likely than not that a pETaG is associated with the gene cluster (e.g., BGC) (“Tier 1”); (3) more likely than not that a pETaG is not associated with the gene cluster (e.g., BGC) (“Tier 2”); and (4) low likelihood that a pETaG is associated with the gene cluster (e.g., BGC) (“Tier 3”). In some embodiments, the heat map and/or one or more subsections thereof may be used as input for the LSTM.

As illustrated in FIG. 4B-1 and FIG. 4B-2, the values of cells in each column of the heat map are sequentialized into a plurality of input arrays each corresponding to a query gene across a number of genomes, and each of the plurality of input arrays is inputted into an LSTM cell.

Each input array also harbors the positional information of the pETaG and the core synthase, represented by two scalars, with 1 and 0 corresponding to the presence and absence of the particular gene (pETaG or core synthase). The plurality of LSTM cells provide an output tier. In some embodiments, subsequent to computing the heat map, vector representations of the data included in the heat map (e.g., a table of values indicating one or more patterns, one or more colors, pETaG position, and so forth) may be provided to one or more neural networks to perform an embeddedness classification of the pETaG in the heat map. For example, in some embodiments, the one or more neural networks may include, for example, a long short-term memory (LSTM) model, convolutional neural network (CNN), or other recurrent neural network (RNN) that may be suitable for processing genomic data or other text-based data expressed as a sequence of elements. For example, in one embodiment, the LSTM model may receive as input linear vector representations of heat map data (e.g., values indicating one or more patterns, one or more colors, pETaG position, and so forth) and output the embeddedness classification (e.g., a probability value of how embedded the pETaG is within a cluster of genes). In some embodiments, the LSTM model perform embeddedness classifications by classifying a pETaG into one of 4 classes: (1) “true positive” (e.g., high likelihood that a pETaG is associated with the BGC (“Tier A+”)); (2) “promising” (e.g., more likely than not that a pETaG is associated with the BGC (“Tier 1”)); (3) “inconclusive” (e.g., more likely than not that a pETaG is not associated with the BGC (“Tier 2”)); and (4) “true negative” (e.g., low likelihood that a pETaG is associated with the BGC (“Tier 3”)).

FIGS. 4B-1 to 4B-2 and FIGS. 4C-1 to 4C-2 illustrate one or more running examples of the LSTM model outputting the embeddedness classification (e.g., a probability value of how embedded the pETaG is within a cluster of genes) based on input linear vector representations of heat map data (e.g., values indicating one or more patterns, one or more colors, pETaG position, and so forth), in accordance with the presently disclosed embodiments. For example, in some embodiments, as depicted by FIGS. 4B-1 to 4B-2, the LSTM model may include a series of memory tiers, each including, for example, a respective memory cell. In some embodiments, the respective memory cells may operate, for example, according to its cell state (e.g., Cₜ₋₁ to Cₜ). The LSTM model may include the ability to remove or add information to the cell state, carefully regulated by structures called gates. In some embodiments, the gates of the respective memory cells may be provided to optionally let information through. For example, in some embodiments, the respective memory cells may include a sigmoid neural net layer and a pointwise multiplication operation. The sigmoid layer outputs numbers between “0” and “1”, describing how much data of each component should be passed through. For example, in one embodiment, a value of “0” means “do not let data pass,” while a value of “1” means “let data pass.” In some embodiments, each respective memory cell may include, for example, these gates to protect and control the cell state.

In some embodiments, during operation, each tier and memory cell of the LSTM model may begin by determining which of the heat map data will be discarded from the cell state. For example, in some embodiments, the determinations may be performed by a sigmoid layer called the forget gate layer, which looks at input data and outputs a number between “0” and “1” for each number in the cell state (e.g., Cₜ₋₁ to Cₜ) in which “1” represents completely keep this data while a “0” represents completely discard this data. The respective tiers and memory cells of the LSTM model may then determine what new information is to be stores in the cell state. For example, in some embodiments, a sigmoid layer called the input gate layer determines which values to update, and a tan h layer creates a vector of new candidate values that could be added to the state.

The respective tiers and memory cells of the LSTM model may then update the old cell state, Cₜ₋₁, into the new cell state Cₜ. The LSTM model may then determine what is to be output based on the cell states of each of the respective tiers and memory cells of the LSTM model. For example, in some embodiments, the sigmoid layer determines what parts of the cell state is to be outputted and then that cell state is passed through the tan h layer to set the values to be between “−1” and “+1” and multiply the values by the output of the sigmoid gate.

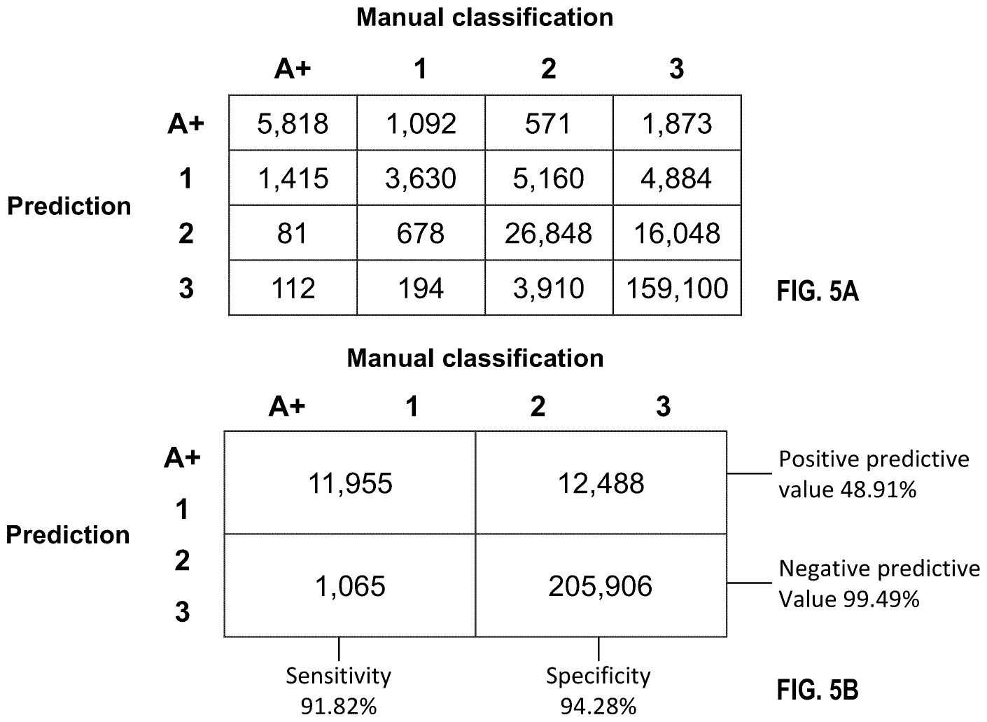

FIGS. 4C-1 to 4C-2 illustrate an example of an output of the LSTM model that, as discussed above, may represent embeddedness classifications of a pETaG into one of 4 classes: (1) “true positive” (e.g., high likelihood that a pETaG is associated with the BGC (“Tier A+”)); (2) “promising” (e.g., more likely than not that a pETaG is associated with the BGC (“Tier 1”)); (3) “inconclusive” (e.g., more likely than not that a pETaG is not associated with the BGC (“Tier 2”)); and (4) “true negative” (e.g., low likelihood that a pETaG is associated with the BGC (“Tier 3”)), as further illustrated by the prediction tables of FIGS. 5A and 5B, in accordance with the presently disclosed embodiments.

Specifically, FIG. 5A illustrates a table of prediction embeddedness benchmark values for “Tier A+”, “Tier 1”, “Tier 2”, and “Tier 3”. Similarly, FIG. 5B illustrates a table of final prediction embeddedness benchmark values for “Tier A+”, “Tier 1”, “Tier 2”, and “Tier 3”, including a positive predictive value (i.e., precision) of 48.91%, a negative predictive value of 99.49%, a sensitivity value of 91.82%, and a specificity value (i.e., recall) of 94.28%. The values in the table of FIG. 5A are calculated from comparing the manually annotated Tier versus the prediction results from the LSTM model. In the table of FIG. 5B, Tier A+ and Tier 1 results are combined and Tier 2 and Tier 3 results are combined. Sensitivity is calculated as the true predicted positives divided by total actual positives. Specificity is calculated as the true predicted negatives divided by the total actual negatives. Positive predictive value is calculated as the true predicted positives divided by the total predicted positives. Negative predictive value is calculated as the true predicted negatives divided by the total predicted negatives.

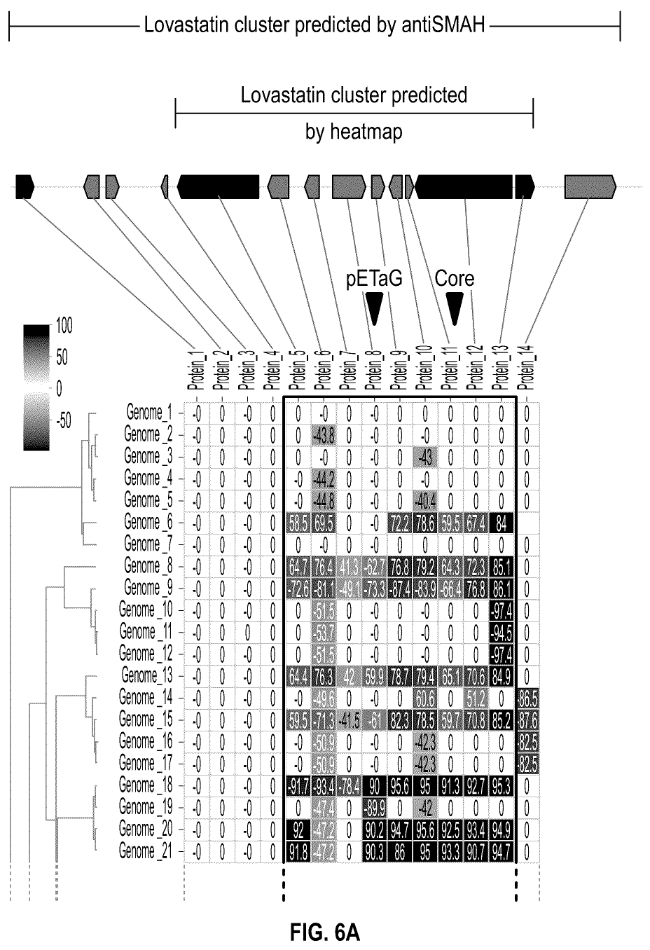

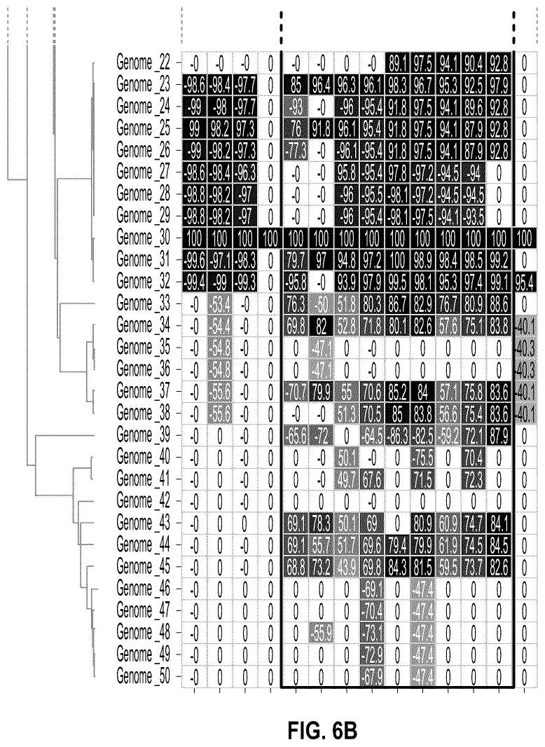

FIGS. 6A and 6B show an example of a BGC for production of lovastatin as predicted by antiSMASH in Aspergillus terreus. Using the methods described herein, a smaller set of genes are identified as being associated with the BGC. FIGS. 7A-1 to 7D-2 show heat maps classified as Tier A+, Tier 1, Tier 2 and Tier 3, respectively.

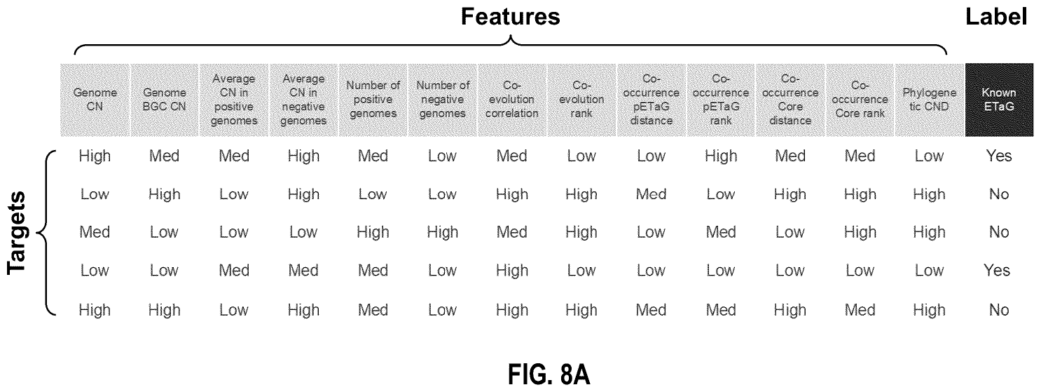

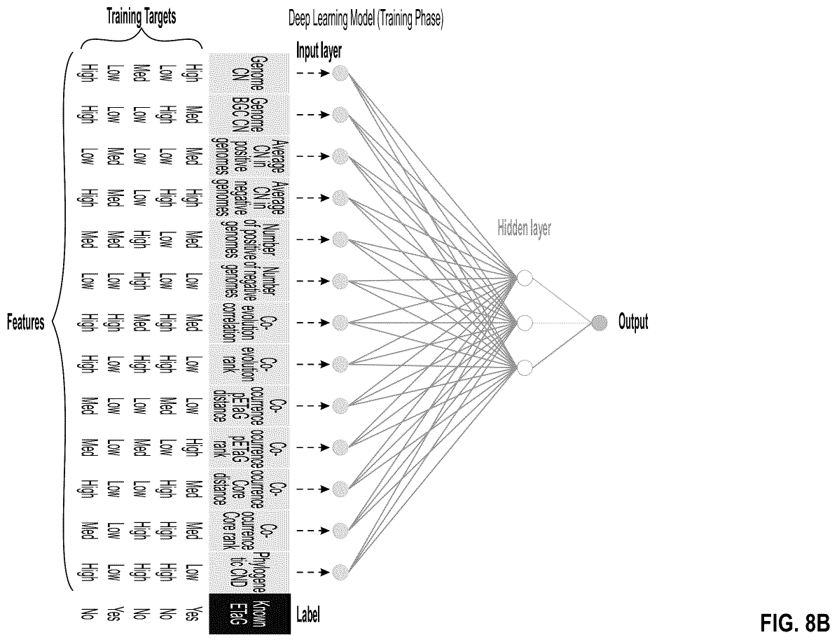

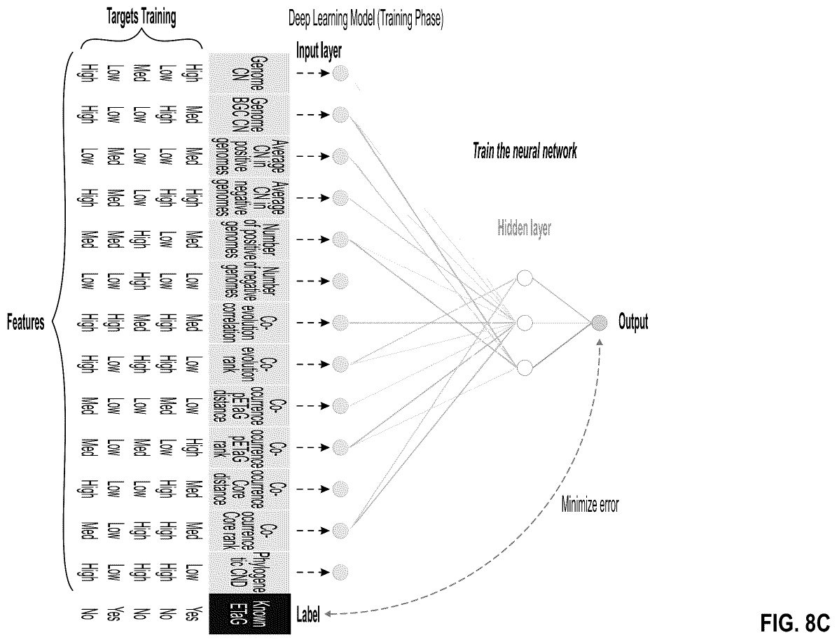

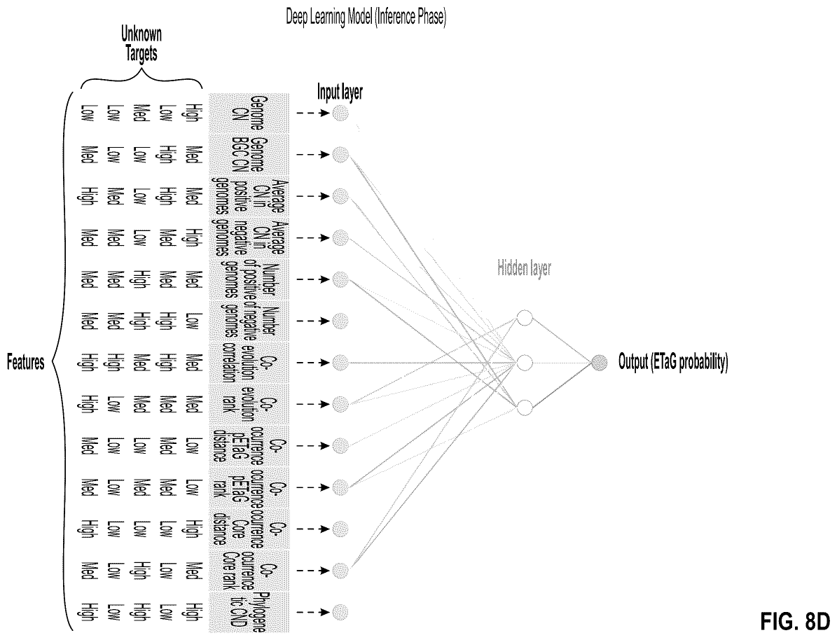

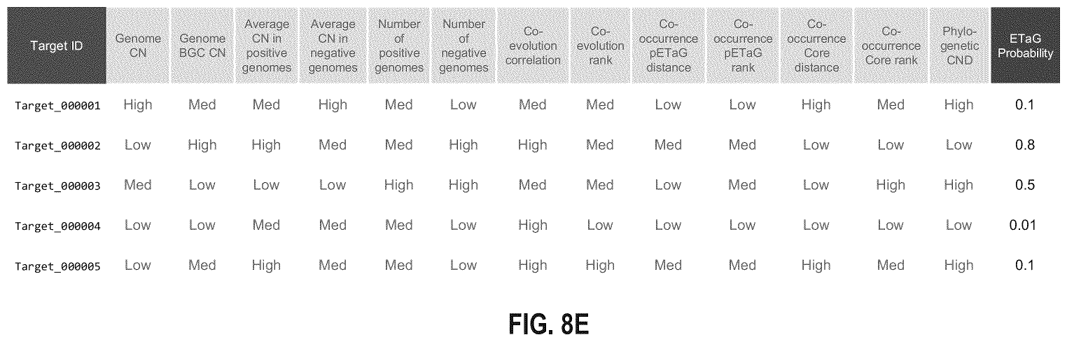

In some embodiments, based on the LSTM model outputting the embeddedness classification (e.g., a probability value of how embedded the pETaG is within a cluster of genes), the output(s) of the LSTM model may be organized into one or more tables (as illustrated by FIG. 8A) representing 4 features “Tier A+”, “Tier 1”, “Tier 2”, and “Tier 3”, and combined with a predetermined set of additional features (e.g., up to 27 features or more including the 4 features “Tier A+”, “Tier 1”, “Tier 2”, and “Tier 3”).

### Machine Learning Model for Predicting ETaG Likelihood

Specifically, FIG. 8A illustrates a data table including the combined set of features. In some embodiments, the combined set of features (e.g., up to 27 features or more) may be organized into a data table as illustrated by FIG. 8A may be utilized to train a neural network (e.g., an artificial neural network (ANN), a multilayer perceptron (MLP), a deep neural network (DNN), a convolutional neural network (CNN), and so forth) to output an ETaG or pETaG probability value based on the inputs of the combined set of features illustrated in FIG. 8A. In some embodiments, the data can be utilized to train other types of machine-learning models (e.g. Bayesian inference, decision tree-based methods such as XGBoost or Random Forest, and so forth). In some embodiments, the data can be utilized to train logistic regression models or other types of supervised models. As further illustrated by FIG. 8A, the data table of the combined set of features may also include an annotated data set of known ETaG or pETaG label values that may be utilized, for example, as a ground truth or other reference for training the neural network.

FIG. 8B illustrates an initial training phase of a neural network trained to output an ETaG or pETaG probability value, in accordance with the presently disclosed embodiments. As illustrated, a training data set corresponding to the features included in the data table of FIG. 8A may be input to an input layer of the neural network. Due to the structured or semi-structured input data, in one embodiment, the neural network may include a multilayer perceptron (MLP) or other layered neural network including at least one hidden layer. For example, during training, the table of features may be input to respective neurons or nodes. Specifically, in some embodiments, the respective neurons or nodes may take as input the table of features and perform one or more designated activation functions (e.g., computational functions) to generate outputs based thereon. For example, in some embodiments, the designated activation functions (e.g., computational functions) may specifically determine the value of the outputs of the input neurons or nodes.

In some embodiments, the respective input neurons or nodes may be connected to a set of hidden neurons or nodes, which may receive the outputs of the input neurons or nodes. In some embodiments, the hidden neurons or nodes may constitute a hidden layer of the neural network, and may each include, for example, a weight that determine the relative strength (e.g., positive or negative) of the input of each connection to the input neurons or nodes. For example, in some embodiments, the hidden layer weights may influence, for example, the effect each input will have on the hidden neurons or nodes and may be adjusted iteratively for the neural network to learn over time. In some embodiments, as further illustrated by FIG. 8B, the neural network may be trained based on, for example, a forward propagation technique. In some embodiments, the forward propagation, and, by extension, the combined outputs of the hidden neurons or nodes, may include the weighted sum of the outputs of the hidden neurons or nodes and the predicted ETaG or pETaG label value (e.g., as compared to the ground truth ETaG or pETaG label value).

FIG. 8C further illustrates the training phase of a neural network trained to output an ETaG or pETaG probability value, in accordance with the presently disclosed embodiments. For example, as depicted by FIG. 8C, a loss function or cost function may be utilized (e.g., supervised learning) to evaluate the neural network by comparing the predicted ETaG or pETaG label value to the ground truth ETaG or pETaG label value to compute a loss (e.g., error). In some embodiments, the weights of the hidden neurons or nodes may be iteratively adjusted, such that the loss (e.g., computed based on the comparison of the predicted ETaG or pETaG label value to the ground truth ETaG or pETaG label) may be minimized to a degree that the neural network is adequately and accurately trained.

FIG. 8D illustrates the inference phase of a neural network trained to output an ETaG or pETaG probability value, in accordance with the presently disclosed embodiments. As illustrated, an unknown data set of features corresponding, for example, to one or more features included in the data table of FIG. 8A, may be input to an input layer of the neural network. For example, during inference, the table of features may be input to respective neurons or nodes. Specifically, as previously discussed with respect to FIG. 8B, the respective neurons or nodes may take as input the table of features and perform one or more designated activation functions (e.g., computational functions) to generate outputs based thereon. For example, in some embodiments, the designated activation functions (e.g., computational functions) may specifically determine the value of the outputs of the input neurons or nodes. In some embodiments, the respective input neurons or nodes may be connected to a set of hidden neurons or nodes, which may receive the outputs of the input neurons or nodes. In some embodiments, the hidden neurons or nodes may each include, for example, a weight that determines the relative strength (e.g., positive or negative) of the input of each connection to the input neurons or nodes. In some embodiments, as further illustrated by FIG. 8D, the combined outputs of the hidden neurons or nodes may include the weighted sum of the outputs of the hidden neurons or nodes and the predicted ETaG or pETaG label value (e.g., as compared to the ground truth ETaG or pETaG label value). Specifically, in accordance with the presently disclosed embodiments, the neural network may output an ETaG or pETaG probability value based on the unknown input data set of features. FIG. 8E illustrates an example data table including the unknown input data set of features and the corresponding outputs of ETaG or pETaG probability values for each of the features, in accordance with the presently disclosed embodiments. In this way, the present embodiments may identify and determine a likelihood that an ETaG or pETaG is associated with one or more features representing BGCs.

### Applications

The computer-based methods described in the “Grid representation analysis methods” section described herein have various applications.

In some embodiments, the present disclosure provides methods and systems for determining borders of a gene cluster (e.g., a BGC) by identifying a plurality of genes associated with the gene cluster (e.g., BGC). In some embodiments, the method comprises: (1) identifying a plurality of query genes that are co-localized with an anchor gene (e.g., a core synthase gene) of a BGC in a query (or reference) genome; (2) for each of the plurality of query genes, performing any one of the computer-implemented methods described in the “Grid representation analysis methods” section for determining a likelihood that the query gene, or an ortholog thereof, is associated with a corresponding BGC in a plurality of genomes; and (c) identifying query genes, or orthologs thereof, having a specified high likelihood of being associated with the BGC as the plurality of genes associated with the BGC, wherein the specified high likelihood is a likelihood beyond a threshold value. For example, if a query gene, or an ortholog thereof, has a probability of more than any one of 30%, 40%, 50%, 60%, 70%, 80%, 90%, or higher for the category of (1) high likelihood, the query gene is associated with the BGC. In some embodiments, if a query gene, or an ortholog thereof, has a combined probability of more than about any one of 50%, 60%, 70%, 80%, 90%, or higher for the categories of (1) high likelihood; and (2) more likely than not, the query gene is associated with the BGC. In some embodiments, if a query gene, or an ortholog thereof, has a probability of more than about any one of 30%, 40%, 50%, 60%, 70%, 80%, 90%, or higher for the category of (4) low likelihood, the query gene is rejected as not being associated with the BGC. The borders (i.e., upstream and downstream limits) of the BGC can be determined based on the location of all genes associated with the BGC as determined using this method.

In some embodiments, the present disclosure provides methods and systems for identifying a resistance gene against a secondary metabolite produced by a BGC in a query (or reference) genome, comprising: (a) identifying a putative embedded gene that is co-localized (e.g., within a proximity zone of no more than about 100 kb, 50 kb, 20 kb, or a user-specified distance) with an anchor gene (e.g., core synthase gene) in the BGC in the query genome, wherein the putative embedded gene is not involved in the production of the secondary metabolite by the BGC; (b) performing any one of the computer-implemented methods described in the “Grid representation analysis methods” section for determining a likelihood that the putative embedded gene is associated with the BGC; and (c) identifying the putative embedded gene as a resistance gene based at least in part on the likelihood that the putative embedded gene is associated with the BGC. In some embodiments, the putative embedded gene is identified as a resistance gene if the likelihood that it is associated with the BGC is above a specified threshold value. In some embodiments, the likelihood that the putative embedded gene is associated with the BGC is one of a plurality of factors used for identifying the putative embedded gene as a resistance gene associated with the BGC. In some embodiments, the method further comprises experimentally verifying that the putative embedded gene is a resistance gene against the secondary metabolite produced by the BGC in the query genome. For example, the putative embedded gene may be expressed and contacted with the secondary metabolite to determine if binding occurs between the product of the putative embedded gene and the secondary metabolite.

In some embodiments, the present disclosure provides methods and systems for mammalian (e.g., human) target identification and/or characterization. For example, a resistance gene (e.g., a fungal resistance gene) identified using the methods described herein, which has a homolog in the human genome, provides a connection between the resistance gene, the human homolog, and the secondary metabolite produced by the BGC. The connection suggests that the human homolog may be a human target of the secondary metabolite, and the secondary metabolite may interact with and/or modulate the human homolog.

In some embodiments, the present disclosure provides methods for identifying and/or characterizing a mammalian (e.g., human) target of a secondary metabolite of a BGC, or an analog of the BGC product, comprising: (1) identifying a putative embedded target gene (pETaG) that is co-localized (e.g., within a proximity zone of no more than about 200 kb, 100 kb, 50 kb, 40 kb, 30 kb, 20 kb or less) with the BGC in a query genome, wherein the pETaG is homologous to a mammalian (e.g., human) gene and the pETaG does not encode an enzyme that produces the secondary metabolite of the BGC; (2) performing any one of the computer-based methods described in the “Grid representation analysis methods” section for determining a likelihood that the pETaG is associated with the BGC; and (3) identifying the mammalian (e.g., human) gene as a target of the secondary metabolite of the BGC based at least in part on the likelihood that the pETaG is associated with the BGC. In some embodiments, the mammalian (e.g., human) gene is identified as a target if the likelihood that it is associated with the BGC is above a threshold value. In some embodiments, the likelihood that the pETaG is associated with the BGC is one of a plurality of factors used for identifying the mammalian (e.g., human) gene as a target of the secondary metabolite of the BGC. In some embodiments, the method further comprises identifying mammalian (e.g., human) homologs of the pETaG in a mammalian (e.g., human) genome. In some embodiments, the method further comprises assaying an effect of the secondary metabolite produced by the BGC, or an analog of the BGC product, on the mammalian (e.g., human) target.

In some embodiments, the present disclosure provides methods and systems for drug discovery, e.g., identifying a small molecule modulator of a mammalian target gene (or a reptilian target gene, avian target gene, amphibian target gene, or a target gene from any other organism). In some embodiments, the present disclosure provides methods for identifying a small molecule modulator of a mammalian (e.g., human) target gene or product thereof, comprising: (a) identifying a homologous gene of the mammalian target gene in a fungal genome (or in an archaea genome, a bacterial genome, a plant genome, or other genomes that comprise BGCs), wherein the homologous gene is co-localized (e.g., within a proximity zone of no more than about 100 kb, 50 kb, 40 kb, 30 kb, 20 kb or less) with an anchor gene (e.g., a core synthase gene) of a BGC of the fungal genome, and wherein the homologous gene is not involved in production of a secondary metabolite by the BGC; (b) performing any one of the computer-based methods described in the “Grid representation analysis methods” section for determining a likelihood that the homologous gene is associated with the BGC; and (c) identifying the secondary metabolite, or an analog thereof, as a small molecule modulator of the mammalian target gene, or product thereof, based at least in part on the likelihood that the homologous gene is associated with the BGC. In some embodiments, the secondary metabolite, or analog thereof, is identified as a small molecule modulator of the mammalian (e.g., human) gene, or product thereof, if the likelihood that the homologous gene is associated with the BGC is above a threshold value. In some embodiments, the likelihood that the homologous gene is associated with the BGC is one of a plurality of factors used for identifying the secondary metabolite, or an analog thereof, as a small molecule modulator of the mammalian (e.g., human) gene or product thereof. In some embodiments, the method further comprises assessing interactions of the mammalian target gene product with a compound derived from the secondary metabolite produced by the BGC. In some embodiments, the method comprises contacting the secondary metabolite, or analog thereof, with a protein encoded by the mammalian target gene, and detecting an activity of the protein encoded by the mammalian target gene. In some embodiments, the activity is binding of the protein encoded by the mammalian target gene with the secondary metabolite, or analog thereof.

In some embodiments, the secondary metabolite is a product of enzymes encoded by the BGC, or a salt thereof, including an unnatural salt. In some embodiments, the secondary metabolite, or analog thereof, is an analog of a product of enzymes encoded by the BGC, e.g., a small molecule compound having the same core structure as the secondary metabolite, or a salt thereof.

In some embodiments, the present disclosure provides methods for modulating a human target, comprising: providing a secondary metabolite produced by enzymes encoded by a BGC, or an analog thereof, wherein the human target (or a nucleic acid sequence encoding the human target) is homologous to an ETaG that is associated with the BGC as determined using any one of the methods described herein.

In some embodiments, the present disclosure provides methods for treating a condition, disorder, or disease associated with a human target, comprising administering to a subject susceptible to, or suffering therefrom, a secondary metabolite produced by enzymes encoded by a BGC, or an analog thereof, wherein the human target (or a nucleic acid sequence encoding the human target) is homologous to an ETaG that is associated with the BGC as determined using any one of the methods described herein.

In some embodiments, the secondary metabolite is produced by a fungus. In some embodiments, the secondary metabolite is acyclic. In some embodiments, the secondary metabolite is a polyketide. In some embodiments, the secondary metabolite is a terpene compound. In some embodiments, the secondary metabolite is a non-ribosomally synthesized peptide.

In some embodiments, an analog of a substance (e.g., secondary metabolite) that shares one or more particular structural features, elements, components, or moieties with a reference substance. Typically, an analog shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance. In some embodiments, an analog of a substance is the substance being substituted at one or more of its substitutable positions.

In some embodiments, an analog of a product comprises the structural core of a product. In some embodiments, a biosynthetic product is cyclic, e.g., monocyclic, bicyclic, or polycyclic, and the structural core of the product is or comprises the monocyclic, bicyclic, or polycyclic ring system. In some embodiments, the structural core of the product comprises one ring of the bicyclic or polycyclic ring system of the product. In some embodiments, a product is or comprises a polypeptide, and a structural core is the backbone of the polypeptide. In some embodiments, a product is or comprises a polyketide, and a structural core is the backbone of the polyketide. In some embodiments, an analog is a substituted biosynthetic product comprising one or more suitable substituents.

### Identification of ETaGs

In some embodiments, the present disclosure provides methods of identifying an embedded target gene (“ETaG”) or a mammalian (e.g., human) target gene (i.e., a (human) gene of interest) corresponding to an ETaG. The present disclosure provides methods for identifying and/or characterizing ETaGs, databases including biosynthetic gene cluster and/or ETaG gene sequences (and optionally relevant annotations), systems for identifying and/or characterizing human target genes corresponding to ETaGs, as well as methods of making and/or using such human target genes and/or systems that contain and/or express them, etc. ETaGs have been described, for example, in WO201955816A1, the contents of which are incorporated herein by reference. The methods described herein provide improved methods of identifying ETaGs that are truly associated with BGCs, and reducing calls of false positive ETaGs identified based on co-localization and/or co-regulation with one or more biosynthetic genes in a BGC in a particular genome.

In some embodiments, the methods described herein are applied to identify ETaGs from fungal genomes. In some embodiments, ETaGs from eukaryotic fungi can bear more similarities to mammalian genes than, for example, their counterparts, if any, in prokaryotes such as certain bacteria. In some embodiments, fungi contain ETaGs that are more therapeutically relevant, and/or contain more therapeutically relevant ETaGs, than organisms that are evolutionarily more distant from human.

In some embodiments, the method comprises: (a) identifying a putative ETaG (pETaG) sequence in a fungal genome, wherein: (1) the pETaG is co-localized (i.e., within a proximity zone relative) to an anchor gene (e.g., core synthase gene) in a BGC; (2) the pETaG is not involved in the production of the secondary metabolite by the BGC; and (3) the pETaG is homologous to an expressed mammalian nucleic acid sequence; (b) determining a likelihood that the pETaG is associated with the BGC using any one of the computer-based methods described in the “Grid representation analysis methods” section; and (c) identifying the pETaG as an ETaG based on the likelihood that the pETaG is associated with the BGC. For example, the pETaG may be identified as an ETaG if the likelihood is above a threshold value, or the likelihood is one of a plurality of factors used for identifying the pETaG as an ETaG. In some embodiments, the pETaG is co-regulated with at least one biosynthetic gene in the BGC. In some embodiments, the pETaG is not co-regulated with at least one biosynthetic gene in the BGC. In some embodiments, the method is repeated for a plurality of pETaGs and used to prioritize the pETaGs for experimental verification based on the likelihoods that the pETaGs are associated with the BGCs. In some embodiments, the ETaG is compared with mammalian, e.g., human, nucleic acid sequences to identify the homologous mammalian nucleic acid sequences. In some embodiments, such a method can be used to identify ETaGs on genomic scales, e.g., from sequences of many (e.g., hundreds, thousands, or even more) genomes. Identified ETaGs can be prioritized based on therapeutic importance of their mammalian homologs, particularly human homologs. In some embodiments, a biosynthetic product (i.e., secondary metabolite) produced by enzymes encoded by the related biosynthetic gene cluster, or an analog thereof, is a modulator (e.g., an activator, an inhibitor, etc.) of a human target. In some embodiments, a biosynthetic product (i.e., secondary metabolite) produced by enzymes encoded by the related biosynthetic gene cluster, or an analog thereof, is a modulator (e.g., an activator, an inhibitor, etc.) of an animal, bacterial, archaca, fungal, or plant target.

As readily appreciated by those skilled in the art, the connection between the biosynthetic products from biosynthetic gene clusters, ETaGs, and human targets, once established, can be utilized in various methods. For example, one may start from a biosynthetic product produced by the enzymes encoded by a biosynthetic gene cluster, to identify an ETaG located within a specified proximity zone of a biosynthetic gene of the biosynthetic gene cluster, and then identify a human target that is homologous to the ETaG. Once the human target is identified, one can prioritize it (even if it was previously considered undruggable), and develop modulators of the human target using the biosynthetic product, including optional further optimization of the biosynthetic product, for medical use, e.g., by preparing and assaying analogs of the product, using any of a variety of methods known to those of skill in the art. One may also start from a human target of therapeutic interest, to identify an ETaG homologous to the human target, and then to identify a biosynthetic gene cluster that contains a biosynthetic gene within a specified proximity zone of the ETaG. Once the biosynthetic gene cluster is identified, the biosynthetic product produced by the enzymes encoded by the biosynthetic gene cluster can be characterized and assayed for modulation of the human target, or a product thereof. The biosynthetic product can be used as a lead compound for optimization of drug candidates using any of a variety of methods known to those of skill in the art to provide agents useful for many medical, e.g., therapeutics purposes. In some embodiments, the target can be from other kingdoms of life such as animals, plants, fungal, bacterial, archaca.

In some embodiments, the present disclosure provides insights particularly into targets that were considered undruggable prior to the present disclosure, by providing methods for identifying their homologous ETaGs in fungi and elucidating the associated biosynthetic gene clusters. In some embodiments, the present disclosure greatly improves druggability of targets that were considered undruggable prior to the present disclosure, in some cases, essentially converting them into druggable targets, by, for example, identifying their homologous ETaGs in fungi, elucidating the associated biosynthetic gene clusters, and testing the biosynthetic products of the related biosynthetic gene clusters (which can be directly used as modulators, and/or whose analogs can be used as modulators, of the human targets).

An ETaG is within a proximity zone relative to an anchor gene (e.g., a core synthase gene) in a BGC, is homologous to an expressed mammalian nucleic acid sequence, and is optionally co-regulated with at least one biosynthetic gene in the BGC. In some embodiments, the ETaG is located not more than about any one of 100 kb, 50 kb, 40 kb, 30 kb, 20 kb, 10 kb, or less from an anchor gene (e.g., a core synthase gene) is a BGC.

In some embodiments, an ETaG is homologous to a human nucleic acid sequence that is, or encodes a product, which is an existing target of therapeutic interest. In some embodiments, an ETaG is homologous to a human nucleic acid sequence that is, or encodes a product, which is a novel target of therapeutic interest. In some embodiments, an ETaG is homologous to a human nucleic acid sequence that is, or encodes a product, which is a target considered undruggable prior to the present disclosure. In some embodiments, an ETaG is homologous to a human nucleic acid sequence that is, or encodes a product, which is a target considered undruggable by small molecules prior to the present disclosure.

In some embodiments, an ETaG sequence is homologous to an expressed mammalian nucleic acid sequence in that its sequence, or a portion thereof, is at least 20%, 30%, 40%, 50%, 60%, 70%, 80%, or 90% identical to that of an expressed mammalian nucleic acid sequence. In some embodiments, an ETaG sequence is homologous to a mammalian nucleic acid sequence in that mRNA produced from an ETaG, or a portion thereof, is homologous to that of a mammalian nucleic acid sequence. In some embodiments, a homologous portion is at least 50, 100, 150, 200, 500, 1000, 2000, 3000, or 5000 base pairs in length. In some embodiments, a homologous portion encodes a conserved protein, or a conserved portion of protein, such as a protein domain, a set of residues that relates to a function (e.g., interaction to another molecule (e.g., a protein, a small molecule, etc.), enzymatic activity, etc.), etc., from fungi to a mammal. In some embodiments, a mammalian nucleic acid, e.g., a human nucleic acid sequence, is related to a human disease, disorder, or condition. In some embodiments, such a human nucleic acid sequence is an existing target of therapeutic interest. In some embodiments, such a human nucleic acid sequence is a novel target of therapeutic interest. In some embodiments, such a human nucleic acid sequence is a target previously considered not susceptible to targeting by, e.g., small molecules.

In some embodiments, an ETaG sequence is homologous to a mammalian nucleic acid sequence in that a product encoded by an ETaG, or a portion thereof, is homologous to that encoded by a mammalian nucleic acid sequence. In some embodiments, an ETaG sequence is homologous to a mammalian nucleic acid sequence in that a protein encoded by an ETaG, or a portion thereof, is homologous to that encoded by a mammalian nucleic acid sequence. In some embodiments, an ETaG sequence is homologous to a mammalian nucleic acid sequence in that a portion of a protein encoded by an ETaG is homologous to that encoded by a mammalian nucleic acid sequence.

In some embodiments, a portion of a protein is a protein domain. In some embodiments, a protein domain is an enzymatic domain. In some embodiments, a protein domain interacts with one or more agents, e.g., small molecules, lipids, carbohydrates, nucleic acids, proteins, etc.

In some embodiments, a portion of a protein is a functional and/or structural domain that defines a protein family that the protein belongs to. Amino acid resides that within specific catalytic or structural domain defining patent families can be selected based on predictive subfamily domain architecture, and optionally verified by various assays, for use in alignment analysis of homology.

In some embodiments, a portion of a protein is a set of key residues, either consecutive or not consecutive, that are important for a function of a protein. In some embodiments, a function is an enzymatic activity, and a portion of a protein is a set of residues that are required for the activity. In some embodiments, a function is an enzymatic activity, and a portion of a protein is a set of residues that interact with a substrate, an intermediate, or a product. In some embodiments, a set of residues interact with a substrate. In some embodiments, a set of residues interact with an intermediate. In some embodiments, a set of residues interact with a product.

In some embodiments, a function of a protein is an interaction with one or more agents, e.g., small molecules, lipids, carbohydrates, nucleic acids, proteins, etc., and a portion of a protein is a set of residues that are required for the interaction. In some embodiments, a set of residues each independently contact an interacting agent. For example, in some embodiments, each of the residues of a set independently contacts an interacting small molecule. In some embodiments, a protein is a kinase and an interacting small molecule is or comprises a nucleobase, and a set of residues each independently contact the nucleobase via, e.g., hydrogen bonding, electrostatic forces, van der Waals forces, aromatic stacking, etc. In some embodiments, an interacting agent is another macromolecule. In some embodiments, an interaction agent is a nucleic acid. In some embodiments, a set of residues are those that contact an interacting nucleic acid, for example, those in transcription factors. In some embodiments, a set of residues are those that contact an interacting protein.

In some embodiments, a portion of a protein is or comprises an essential structural element of protein effector recruitment and/or binding, for example, based on tertiary protein structures of human targets.

Portions of proteins, such as protein domains, sets of residues responsible for biological functions, etc., can be conserved from species to species, for example, in some embodiments from fungi to human as illustrated in the present disclosure.

In some embodiments, protein homology is measured based on exact identity, e.g., the same amino acid residues at given positions. In some embodiments, homology is measured based on one or more properties, e.g., amino acid residues bearing one or more identical or similar properties (e.g., polar, non-polar, hydrophobic, hydrophilic, size, acidic, basic, aromatic, etc.). Exemplary methods for assessing homology are widely known in the art and can be utilized in accordance with the present disclosure, for example, MAFFT, MUSCLE, TCoffee, ClustalW, etc.

In some embodiments, a protein encoded by an ETaG, or a portion thereof (e.g., those described in the present disclosure), is at least 20%, 30%, 40%, 50%, 60%, 70%, 80%, 85%, 90%, 91%, 92%, 93%, 94%, 95%, 96%, 97%, 98%, or 99%, or 100% (when 100% it is identical) homologous to that encoded by a mammalian nucleic acid sequence. In some embodiments, a protein encoded by an ETaG, or a portion thereof, is at least 50%, 60%, 70%, 80%, 85%, 90%, 91%, 92%, 93%, 94%, 95%, 96%, 97%, 98%, or 99%, or 100% homologous to that encoded by an expressed mammalian nucleic acid sequence.

In some embodiments, an ETaG is co-regulated with at least one biosynthetic gene in the biosynthetic gene cluster. In some embodiments, an ETaG is co-regulated with two or more genes in the biosynthetic gene cluster. In some embodiments, an ETaG is co-regulated with the biosynthetic gene cluster in that expression of the ETaG is increased, or turned on, when a biosynthetic product produced by the enzymes encoded by the biosynthetic gene cluster (a biosynthetic product of the biosynthetic gene cluster) is produced. In some embodiments, an ETaG is co-regulated with the biosynthetic gene cluster in that expression of the ETaG is increased, or turned on, when level of a biosynthetic product of the biosynthetic gene cluster is increased.

In some embodiments, an organism comprising an ETaG comprises one or more homologous genes of the ETaG. In some embodiments, an ETaG gene sequence is optionally more than about 10%, 20%, 30%, 40%, 50%, 60%, 70%, 80%, 85%, 90%, 95%, or 99% homologous to one or more gene sequences in the same genome. In some embodiments, an ETaG gene sequence is optionally more than about 10%, 20%, 30%, 40%, 50%, 60%, 70%, 80%, 85%, 90%, 95%, or 99 homologous to 2, 3, 4, 5, 6, 7, 8, 9 or more gene sequences in the same genome. In some embodiments, the homology is more than 10%. In some embodiments, the homology is more than 20%. In some embodiments, the homology is more than 30%. In some embodiments, the homology is more than 40%. In some embodiments, the homology is more than 50%. In some embodiments, the homology is more than 60%. In some embodiments, the homology is more than 70%. In some embodiments, the homology is more than 80%. In some embodiments, the homology is more than 90%.

In some embodiments, an ETaG gene sequence is optionally no more than about 10%, 20%, 30%, 40%, 50%, 60%, 70%, 80%, 85%, 90%, 95%, or 99% identical to any expressed gene sequence in at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% fungal nucleic acid sequence in the set that is from a different fungal strain and comprises a homologous biosynthetic gene cluster. In some embodiments, an ETaG gene sequence is optionally no more than about 10%, 20%, 30%, 40%, 50%, 60%, 70%, 80%, 85%, 90%, 95%, or 99% identical to at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% fungal gene sequence that is within a proximity zone relative to a biosynthetic gene of a homologous biosynthetic gene cluster from a different fungal strain. In some embodiments, an ETaG gene sequence is optionally no more than about 10%, 20%, 30%, 40%, 50%, 60%, 70%, 80%, 85%, 90%, 95%, or 99% identical to at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% fungal gene sequence that is within a proximity zone relative to a biosynthetic gene of a homologous biosynthetic gene cluster from a different fungal strain. In some embodiments, an ETaG gene sequence is optionally no more than about 10%, 20%, 30%, 40%, 50%, 60%, 70%, 80%, 85%, 90%), 95%), or 99% identical to any expressed gene sequence in any fungal nucleic acid sequence in the set that is from a different fungal strain and comprises a homologous biosynthetic gene cluster. In some embodiments, an ETaG gene sequence is optionally no more than about 10%, 20%, 30%, 40%, 50%, 60%, 70%, 80%, 85%, 90%, 95%, or 99% identical to any expressed gene sequence that is within a proximity zone relative to a biosynthetic gene of a homologous biosynthetic gene cluster from a different fungal strain. In some embodiments, it is no more than about 10%) identical. In some embodiments, it is no more than about 20% identical. In some embodiments, it is no more than about 30% identical. In some embodiments, it is no more than about 40%) identical. In some embodiments, it is no more than about 50% identical. In some embodiments, it is no more than about 60% identical. In some embodiments, it is no more than about 70%) identical. In some embodiments, it is no more than about 80% identical. In some embodiments, it is no more than about 90% identical.

In some embodiments, a human target gene and/or a product thereof is susceptible to modulation by a biosynthetic product, or an analog thereof, of a biosynthetic gene cluster, wherein the human target gene has its homologous ETaGs embedded within the biosynthetic gene cluster or located in a specified proximity zone relative to a biosynthetic gene of the cluster. In some embodiments, a protein encoded by a human target gene is susceptible to modulation by a biosynthetic product, or an analog thereof, of a biosynthetic gene cluster, wherein the human target gene has its homologous ETaGs embedded within the biosynthetic gene cluster or located in a specified proximity zone relative to a biosynthetic gene of the cluster. Thus, in some embodiments, the present disclosure not only provides novel human target, but also provides methods and agents for modulating such human targets. In some embodiments, a compound produced by the enzymes of a biosynthetic gene cluster interacts and/or modulates with a target encoded by a mammalian, e.g., human, nucleic sequence that is homologous to an ETaG related to the biosynthetic gene cluster.

In some embodiments, the present disclosure provides methods for assessing compounds using identified ETaGs and products encoded thereby. In some embodiments, the present disclosure provides a method comprising: contacting at least one test compound with a gene product encoded by an embedded target gene of a fungal nucleic acid sequence, and determining that: a level or activity of the gene product is altered when the test compound is present as compared with when it is absent; or a level or activity of the gene product is comparable to that observed when a reference agent having a known effect on the level or activity is present.

In some embodiments, the present disclosure provides methods for identifying and/or characterizing a mammalian, e.g., human, target of a product produced by enzymes encoded by a biosynthetic gene cluster, or an analog of the product, comprising: identifying a human homolog of an ETaG that is determined to be associated with the BGC using any one of the methods described herein; and optionally assaying an effect of the product produced by enzymes encoded by a biosynthetic gene cluster, or an analog of the product, on the target.

Additional analysis can include assessing conservation/similarity of essential structural elements of protein effector recruitment/binding, for example, based on the examination of the tertiary protein structure of the human target. For example, in some embodiments, aligned sequences were compared to the PDB crystal structure. In some embodiments, only amino acids within the specific catalytic or structural domain defining the PFAM boundaries of the ETaG/target (e.g., based on predictive subfamily domain architecture) were used in an alignment analysis. The ETaG sequences were directly compared to their human counterparts by aligning all ETaGs and human target protein(s), with their phylogenetic relationships yielding quantitative correlative data (e.g. peptide sequence similarity and/or evolutionary tree visualization) corresponding to the target protein residues within 4 Angstrom of the corresponding engaging proteins.

Without the intention to be limited by any theory, in cases where these structural motifs are conserved within fungal ETaGs, it may indicate an increased probability that the metabolite produced by the ETaG-related biosynthetic gene cluster is an effector of both fungal and human target proteins, and the metabolite produced can be a drug candidate, or a lead for drug development, toward the human target. In some embodiments, the above analyses are used to prioritize ETaGs and their related biosynthetic gene clusters, and metabolites produced from the biosynthetic gene clusters, with respect to targeting human targets.

### Computer Systems

In some embodiments, provided computer-based methods, sequences, genomes and/or databases are embodied in a computer readable medium. In some embodiments, the present disclosure provides systems comprising one or more non-transitory machine-readable storage media storing data representing provided computer-based methods, sequences, genomes and/or databases. Non-transitory machine-readable storage media suitable for embodying provided data include all forms of non-volatile storage area, including by way of example, semiconductor storage area devices, e.g., EPROM, EEPROM, and flash storage area devices; magnetic disks, e.g., internal hard disks or removable disks; magneto-optical disks; and CD-ROM and DVD-ROM disks. Among other things, provided systems can be particularly efficient due to provided sets and databases having particular structures described herein.

In some embodiments, the present disclosure provides computer systems that can perform provided methods described herein. In some embodiments, the present disclosure provides computer systems adapted to perform provided methods. In some embodiments, the present disclosure provides computer systems adapted to query provided genomes and/or databases. In some embodiments, the present disclosure provides computer systems adapted to access provided databases.

Computer systems that may be used to implement all or part of provided technologies may include various forms of digital computers. Examples of digital computers include, but are not limited to, laptops, desktops, workstations, personal digital assistants, servers, blade servers, mainframes, smart televisions and other appropriate computers. Mobile devices may be used to implement all or part of provided technologies. Mobile devices include, but are not limited to, tablet computing devices, personal digital assistants, cellular telephones, smartphones, digital cameras, digital glasses and other portable computing devices. The computing devices described herein, their connections and relationships, and their functions, are meant to be examples only, and are not meant to limit implementations of the technology.

All or part of technologies described herein and their various modifications can be implemented, at least in part, via a computer program product, e.g., a computer program tangibly embodied in one or more information carriers, e.g., in one or more tangible machine-readable storage media, for execution by, or to control the operation of, data processing apparatus, e.g., a programmable processor, a computer, or multiple computers.

A computer program for provided technologies can be written in any form of programming language, including compiled or interpreted languages, and it can be deployed in any form, including as a stand-alone program or as a module, part, subroutine, or other unit suitable for use in a computing environment. A computer program can be deployed to be executed on one computer or on multiple computers at one site or distributed across multiple sites and interconnected by a network.

Actions, e.g., associated with implementing programs and technologies, can be performed by one or more programmable processors executing one or more computer programs to perform provided technologies. All or part of the processes can be implemented as, special purpose logic circuitry, e.g., an FPGA (field programmable gate array) and/or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only storage area or a random access storage area or both. Elements of a computer (including a server) include one or more processors for executing instructions and one or more storage area devices for storing instructions and data. Generally, a computer will also include, or be operatively coupled to receive data from, or transfer data to, or both, one or more machine-readable storage media, such as mass storage devices for storing data, e.g., magnetic, magneto-optical disks, or optical disks. Non-transitory machine-readable storage media suitable for embodying computer program instructions and data include all forms of non-volatile storage area, including by way of example, semiconductor storage area devices, e.g., EPROM, EEPROM, and flash storage area devices; magnetic disks, e.g., internal hard disks or removable disks; magneto-optical disks; and CD-ROM and DVD-ROM disks.

Each computing device, such as a tablet computer, may include a hard drive for storing data and computer programs, and a processing device (e.g., a microprocessor) and memory (e.g., RAM) for executing computer programs. Each computing device may include an image capture device, such as a still camera or video camera. The image capture device may be built-in or simply accessible to the computing device.

Each computing device may include a graphics system, including a display screen. A display screen, such as an LCD or a CRT (Cathode Ray Tube) displays, to a user, images that are generated by the graphics system of the computing device. As is well known, display on a computer display (e.g., a monitor) physically transforms the computer display. For example, if the computer display is LCD-based, the orientation of liquid crystals can be changed by the application of biasing voltages in a physical transformation that is visually apparent to the user. As another example, if the computer display is a CRT, the state of a fluorescent screen can be changed by the impact of electrons in a physical transformation that is also visually apparent. Each display screen may be touch-sensitive, allowing a user to enter information onto the display screen via a virtual keyboard. On some computing devices, such as a desktop or smartphone, a physical QWERTY keyboard and scroll wheel may be provided for entering information onto the display screen. Each computing device, and computer programs executed thereon, may also be configured to accept voice commands, and to perform functions in response to such commands.

### EXEMPLARY EMBODIMENTS

Among the provided embodiments are:
1. A computer-implemented method, comprising:
   a) identifying a putative gene cluster comprising a putative embedded gene in a query genome from a plurality of genomes, wherein the putative gene cluster comprises an anchor gene known to associate with the gene cluster, and wherein the anchor gene is co-localized with the putative embedded gene;
   b) identifying a plurality of positive genomes comprising an ortholog of the anchor gene and a plurality of negative genomes that do not comprise an ortholog of the anchor gene, wherein the plurality of positive genomes have pairwise sequence similarities of no more than a threshold value, and wherein the plurality of negative genomes are selected based on sequence similarities or phylogenetic distances to the plurality of positive genomes; and
   c) creating a grid representation comprising a plurality of cells arranged according to a first axis and a second axis, wherein the first axis corresponds to all protein-coding genes that are co-localized with the anchor gene in the putative BGC in the query genome, and the second axis corresponds to the plurality of positive genomes and the plurality of negative genomes, wherein each cell is based on:
      (1) the presence or absence of an ortholog of the respective protein-coding gene in the respective genome;
      (2) sequence similarity of the ortholog to the respective protein-coding gene; and
      (3) whether the ortholog of the respective protein-coding gene is co-localized with the ortholog of the anchor gene in the respective genome.
2. A computer-implemented method for determining a likelihood that a putative embedded gene is associated with a gene cluster, wherein the putative embedded gene is co-localized with an anchor gene known to associate with the gene cluster in a query genome, comprising:
   a) receiving a grid representation comprising a plurality of cells arranged according to a first axis and a second axis, wherein the first axis corresponds to a plurality of different genomes, wherein the plurality of genomes comprises a plurality of positive genomes each having an ortholog of the anchor gene and a plurality of negative genomes that do not have an ortholog of the anchor gene, wherein the second axis corresponds to a plurality of query gene orthologs that are co-localized with the anchor gene of the BGC in the query genome, wherein the putative embedded gene is one of the plurality of query genes, and wherein each cell is based on:
      (i) the presence or absence of an ortholog of the respective query gene in the respective genome;
      (ii) sequence similarity of the ortholog to the respective query gene; and
      (iii) whether the ortholog of the respective query gene is co-localized with the ortholog of the anchor gene in the respective genome; and
   b) inputting the grid representation, or a subsection thereof, into a machine-learning model, wherein the machine-learning model is trained to determine a likelihood that the putative embedded gene is embedded in the gene cluster based on values of the plurality of cells in the grid representation, thereby providing the likelihood that the putative embedded gene is associated with the gene cluster.
3. The computer-implemented method of embodiment 2, further comprising generating the grid representation.
4. The computer-implemented method of embodiment 3, wherein generating the grid representation comprises:
   a) identifying a putative gene cluster comprising a putative embedded gene in a query genome from a plurality of genomes, wherein the putative gene cluster comprises an anchor gene known to associate with the gene cluster, and wherein the anchor gene is co-localized with the putative embedded gene;
   b) identifying a plurality of positive genomes comprising an ortholog of the anchor gene and a plurality of negative genomes that do not comprise an ortholog of the anchor gene, wherein the plurality of positive genomes have pairwise sequence similarities of no more than a threshold value, and wherein the plurality of negative genomes are selected based on sequence similarities or phylogenetic distances to the plurality of positive genomes; and
   c) creating a grid representation comprising a plurality of cells arranged according to a first axis and a second axis, wherein the first axis corresponds to all protein-coding genes that are co-localized with the anchor gene in the putative gene cluster in the query genome, and the second axis corresponds to the plurality of positive genomes and the plurality of negative genomes, wherein each cell is based on:
      (1) the presence or absence of an ortholog of the respective protein-coding gene in the respective genome;
      (2) sequence similarity of the ortholog to the respective protein-coding gene; and
      (3) whether the ortholog of the respective protein-coding gene is co-localized with the ortholog of the anchor gene in the respective genome.
5. The computer-implemented method of embodiment any one of embodiments 2-4, wherein the machine-learning model is a classification model configured to output a probability for each of a plurality of predefined likelihood categories.
6. The computer-implemented method of embodiment 5, wherein the classification model is a long short-term memory (LSTM) model or a convolutional neural network (CNN) model.
7. The computer-implemented method of embodiment 5 or 6, wherein the plurality of predefined likelihood categories comprise: (1) high likelihood; (2) more likely than not; (3) more unlikely than not; and (4) low likelihood.
8. The computer-implemented method of any one of the preceding embodiments, wherein the grid representation is a heat map representation.
9. The computer-implemented method of any one of embodiments 2-8, further comprising displaying the grid representation and the likelihood.
10. The computer-implemented method of any one of the preceding embodiments, wherein the grid representation is hierarchically clustered.
11. The computer-implemented method of any one of the preceding embodiments, wherein the number of the positive genomes is equal to the number of negative genomes.
12. The computer-implemented method of embodiment 10 or 11, wherein the plurality of positive genomes is selected from a plurality of genome clusters based on sequence similarities of genomes in a database, wherein no two positive genomes in the grid representation belong to the same genome cluster.
13. The computer-implemented method of embodiment 12, wherein each negative genome is selected by identifying a genome in the database that has the highest sequence similarity or shortest phylogenetic distance to a positive genome, but does not have an ortholog of the anchor gene.
14. The computer-implemented method of any one of the preceding embodiments, wherein the average pairwise percentage sequence identities of orthologs of one or more single copy genes in the positive genomes are no more than about 95%, and/or the average pairwise percentage sequence identities of orthologs of one or more single copy genes in the negative genomes are no more than about 95%.
15. The computer-implemented method of any one of the preceding embodiments, wherein the first axis corresponds to at least 20 genomes.
16. The computer-implemented method of embodiment 15, wherein the first axis corresponds to about 50 genomes.
17. The computer-implemented method of any one of embodiments 1-16, wherein the plurality of genomes are fungal genomes.
18. The computer-implemented method of any one of embodiments 1-16, wherein the plurality of genomes are plant genomes.
19. The computer-implemented method of any one of embodiments 1-16, wherein the plurality of genomes are bacterial genomes.
20. The computer-implemented method of any one of the preceding embodiments, wherein whether a gene is co-localized with an anchor gene of a gene cluster is determined using antiSMASH.
21. The computer-implemented method of any one of the preceding embodiments, wherein whether a gene is co-localized with an anchor gene of a gene cluster is determined based on whether the gene is located within a proximity zone upstream or downstream from the anchor gene.
22. The computer-implemented method of embodiment 21, wherein the proximity zone is no more than 50 kb.
23. The computer-implemented method of embodiment 22, wherein the proximity zone is about 20 kb.
24. The computer-implemented method of any one of embodiments 1-23, wherein the gene cluster is a biosynthetic gene cluster (BGC).
25. A computer-implemented method for identifying a resistance gene against a secondary metabolite produced by a BGC in a query genome, comprising:
   (a) identifying a putative embedded gene co-localized with an anchor gene in the BGC in the query genome, wherein the putative embedded gene is not involved in the production of the secondary metabolite by the BGC;
   (b) performing the method of any one of embodiments 2-24 to determine the likelihood that the putative embedded gene is associated with the BGC; and
   (c) identifying the putative embedded gene as a resistance gene based at least in part on the likelihood that the putative embedded gene is associated with the BGC.
26. A computer-implemented method for identifying a small molecule modulator of a target gene, comprising:
   (a) identifying a homologous gene of the target gene in a fungal genome, wherein the homologous gene is co-localized with an anchor gene a BGC of the fungal genome, and wherein the homologous gene is not involved in production of a secondary metabolite by the BGC;
   (b) performing the method of any one of embodiments 2-24 to determine the likelihood that the homologous gene is associated with the BGC; and
   (c) identifying the secondary metabolite or an analog thereof as a small molecule modulator of the target gene based at least in part on the likelihood that the homologous gene is associate with the BGC.
27. The computer-implemented method of embodiment 26, wherein the homologous gene encodes a protein having at least about 30% sequence identity to a protein encoded by the target gene.
28. The computer-implemented method of embodiment 26 or 27, further comprising contacting the secondary metabolite or analog thereof with a protein encoded by the target gene, and detecting an activity of the protein encoded by the target gene.
29. The computer-implemented method of any one of embodiments 26 to 28, wherein the target gene is a mammalian gene.
30. The computer-implemented method of embodiment 29, wherein the mammalian gene is a human gene.
31. The computer-implemented method of any one of embodiments 26 to 28, wherein the target gene is a reptilian gene, an avian gene, or an amphibian gene.
32. The computer-implemented method of any one of embodiments 26 to 28, wherein the target gene is a bacterial gene.
33. The computer-implemented method of any one of embodiments 26 to 28, wherein the target gene is a fungal gene.
34. The computer-implemented method of any one of embodiments 26 to 28, wherein the target gene is a plant gene.
35. A computer-implemented method of identifying a plurality of genes associated with a BGC, comprising:
   (a) identifying a plurality of query genes that are co-localized with an anchor gene of a BGC in a query genome;
   (b) for each of the plurality of query genes, determining a likelihood that the respective query gene is associated with the BGC using the method of any one of embodiments 2-24; and
   (c) identifying query genes having a high likelihood of being associated with the BGC as the plurality of genes associated with the BGC, wherein the high likelihood is a likelihood that is greater than a threshold value.
36. The computer-implemented method of any one of embodiments 2 to 35, wherein a query gene is a gene that is homologous to a mammalian protein.
37. The computer-implemented method of embodiment 36, wherein the mammalian protein is a human protein.
38. The computer-implemented method of any one of embodiments 2 to 35, wherein a query gene is a gene that is homologous to a reptilian protein, an avian protein, or an amphibian protein.
39. The computer-implemented method of any one of embodiments 2 to 35, wherein a query gene is a gene corresponding to a bacterial protein.
40. The computer-implemented method of any one of embodiments 2 to 35, wherein a query gene is a gene corresponding to a fungal protein.
41. The computer-implemented method of any one of embodiments 2 to 35, wherein a query gene is a gene corresponding to a plant protein.
42. The computer-implemented method of any one of embodiments 1-41, wherein the anchor gene is a core synthase gene of the BGC.
43. A system comprising:
   one or more processors; and
   a memory communicatively coupled to the one or more processors and configured to store instructions that, when executed by the one or more processors, cause the system to perform the method of any one of embodiments 1 to 42.
44. A non-transitory computer-readable storage medium storing one or more programs, the one or more programs comprising instructions, which when executed by one or more processors of an electronic device, cause the electronic device to perform the method of any one of embodiments 1 to 42.

The foregoing description, for the purpose of explanation, has been described with reference to specific examples or aspects. However, the illustrative discussions above are not intended to be exhaustive or to limit the invention to the precise forms disclosed. For the purpose of clarity and a concise description, features are described herein as part of the same or separate variations; however, it will be appreciated that the scope of the disclosure includes variations having combinations of all or some of the features described. Many modifications and variations are possible in view of the above teachings. The variations were chosen and described in order to best explain the principles of the techniques and their practical applications. Others skilled in the art are thereby enabled to best utilize the techniques and various variations with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. (canceled)

2. A computer-implemented method for determining a likelihood that a putative embedded gene is associated with a gene cluster, wherein the putative embedded gene is co-localized with an anchor gene known to associate with the gene cluster in a query genome, comprising:
a) receiving a grid representation comprising a plurality of cells arranged according to a first axis and a second axis, wherein the first axis corresponds to a plurality of different genomes, wherein the plurality of genomes comprises a plurality of positive genomes each having an ortholog of the anchor gene and a plurality of negative genomes that do not have an ortholog of the anchor gene, wherein the second axis corresponds to a plurality of query gene orthologs that are co-localized with the anchor gene of the BGC in the query genome, wherein the putative embedded gene is one of the plurality of query genes, and wherein each cell is based on:
(i) the presence or absence of an ortholog of the respective query gene in the respective genome;
(ii) sequence similarity of the ortholog to the respective query gene; and
(iii) whether the ortholog of the respective query gene is co-localized with the ortholog of the anchor gene in the respective genome; and
b) inputting the grid representation, or a subsection thereof, into a machine-learning model, wherein the machine-learning model is trained to determine a likelihood that the putative embedded gene is embedded in the gene cluster based on values of the plurality of cells in the grid representation, thereby providing the likelihood that the putative embedded gene is associated with the gene cluster.

3. The computer-implemented method of claim 2, further comprising generating the grid representation.

4. The computer-implemented method of claim 3, wherein generating the grid representation comprises:
a) identifying a putative gene cluster comprising a putative embedded gene in a query genome from a plurality of genomes, wherein the putative gene cluster comprises an anchor gene known to associate with the gene cluster, and wherein the anchor gene is co-localized with the putative embedded gene;
b) identifying a plurality of positive genomes comprising an ortholog of the anchor gene and a plurality of negative genomes that do not comprise an ortholog of the anchor gene, wherein the plurality of positive genomes have pairwise sequence similarities of no more than a threshold value, and wherein the plurality of negative genomes are selected based on sequence similarities or phylogenetic distances to the plurality of positive genomes; and
c) creating a grid representation comprising a plurality of cells arranged according to a first axis and a second axis, wherein the first axis corresponds to all protein-coding genes that are co-localized with the anchor gene in the putative gene cluster in the query genome, and the second axis corresponds to the plurality of positive genomes and the plurality of negative genomes, wherein each cell is based on:
(1) the presence or absence of an ortholog of the respective protein-coding gene in the respective genome;
(2) sequence similarity of the ortholog to the respective protein-coding gene; and
(3) whether the ortholog of the respective protein-coding gene is co-localized with the ortholog of the anchor gene in the respective genome.

5. The computer-implemented method of claim 2, wherein the machine-learning model is a classification model configured to output a probability for each of a plurality of predefined likelihood categories.

6. The computer-implemented method of claim 5, wherein the classification model is a long short-term memory (LSTM) model or a convolutional neural network (CNN) model.

7. The computer-implemented method of claim 5, wherein the plurality of predefined likelihood categories comprise: (1) high likelihood; (2) more likely than not; (3) more unlikely than not; and (4) low likelihood.

8. The computer-implemented method of claim 2, wherein the grid representation is a heat map representation.

9. The computer-implemented method of claim 2, further comprising displaying the grid representation and the likelihood.

10. The computer-implemented method of claim 2, wherein the grid representation is hierarchically clustered.

11. The computer-implemented method of claim 2, wherein the number of the positive genomes is equal to the number of negative genomes.

12. The computer-implemented method of claim 10, wherein the plurality of positive genomes is selected from a plurality of genome clusters based on sequence similarities of genomes in a database, wherein no two positive genomes in the grid representation belong to the same genome cluster.

13. The computer-implemented method of claim 12, wherein each negative genome is selected by identifying a genome in the database that has the highest sequence similarity or shortest phylogenetic distance to a positive genome, but does not have an ortholog of the anchor gene.

14. The computer-implemented method of claim 4, wherein the average pairwise percentage sequence identities of orthologs of one or more single copy genes in the positive genomes are no more than about 95%, and/or the average pairwise percentage sequence identities of orthologs of one or more single copy genes in the negative genomes are no more than about 95%.

15. The computer-implemented method of claim 2, wherein the first axis corresponds to at least 20 genomes.

16. The computer-implemented method of claim 15, wherein the first axis corresponds to about 50 genomes.

17. The computer-implemented method of claim 2, wherein the plurality of genomes are fungal genomes.

18. The computer-implemented method of claim 2, wherein the plurality of genomes are plant genomes.

19. The computer-implemented method of claim 2, wherein the plurality of genomes are bacterial genomes.

20. The computer-implemented method of claim 2, wherein whether a gene is co-localized with an anchor gene of a gene cluster is determined using antiSMASH.

21. The computer-implemented method of claim 2, wherein whether the putative embedded gene is co-localized with an anchor gene of a gene cluster is determined based on whether the gene is located within a proximity zone upstream or downstream from the anchor gene.

22. The computer-implemented method of claim 21, wherein the proximity zone is no more than 50 kb.

23. The computer-implemented method of claim 22, wherein the proximity zone is about 20 kb.

24. The computer-implemented method of claim 2, wherein the gene cluster is a biosynthetic gene cluster (BGC).

25. A computer-implemented method for identifying a resistance gene against a secondary metabolite produced by a BGC in a query genome, comprising:
(a) identifying a putative embedded gene co-localized with an anchor gene in the BGC in the query genome, wherein the putative embedded gene is not involved in the production of the secondary metabolite by the BGC;
(b) performing the method of claim 2 to determine the likelihood that the putative embedded gene is associated with the BGC; and
(c) identifying the putative embedded gene as a resistance gene based at least in part on the likelihood that the putative embedded gene is associated with the BGC.

26. A computer-implemented method for identifying a small molecule modulator of a target gene, comprising:
(a) identifying a homologous gene of the target gene in a fungal genome, wherein the homologous gene is co-localized with an anchor gene a BGC of the fungal genome, and wherein the homologous gene is not involved in production of a secondary metabolite by the BGC;
(b) performing the method of claim 2 to determine the likelihood that the homologous gene is associated with the BGC; and
(c) identifying the secondary metabolite or an analog thereof as a small molecule modulator of the target gene based at least in part on the likelihood that the homologous gene is associated with the BGC.

27. The computer-implemented method of claim 26, wherein the homologous gene encodes a protein having at least about 30% sequence identity to a protein encoded by the target gene.

28. The computer-implemented method of claim 26, further comprising contacting the secondary metabolite or analog thereof with a protein encoded by the target gene, and detecting an activity of the protein encoded by the target gene.

29. The computer-implemented method of claim 26, wherein the target gene is a mammalian gene.

30. The computer-implemented method of claim 29, wherein the mammalian gene is a human gene.

31-42. (canceled)

43. A system comprising:
one or more processors; and
a memory communicatively coupled to the one or more processors and configured to store instructions that, when executed by the one or more processors, cause the system to perform the method of claim 2.

44. A non-transitory computer-readable storage medium storing one or more programs, the one or more programs comprising instructions, which when executed by one or more processors of an electronic device, cause the electronic device to perform the method of claim 2.
